(19) United States
(12) Patent Application Publication
(10) Pub. No.: US 2026/0240656 A1
(43) Pub. Date: Aug. 20, 2026

(54) SYSTEMS AND METHODS FOR GUIDING AND POSITIONING AN IMPLANT

(71) Applicant: Edwards Lifesciences Innovation (Israel) Ltd., Caesarea (IL)

(72) Inventors: Gidon Pesach, Kfar Vitkin (IL); Or Cohen, Modi'in (IL); Yaron Herman, Givat Ada (IL); Haim Brauon, Beit Dagan (IL); Aviv Galon, Tel Aviv (IL); Eyal German, Tel Aviv (IL); Ido Avrahamov, Tel-Aviv (IL); Adi White Zeira, Karnei Shomron (IL); Daniela Shabodyash, Atlit (IL)

(21) Appl. No.: 19/656,125

(22) Filed: Apr. 23, 2026

Related U.S. Application Data
(63) Continuation of application No. PCT/IB2024/059287, filed on Sep. 24, 2024.
(60) Provisional application No. 63/593,935, filed on Oct. 27, 2023.

(51) Int. Cl. A61F 2/24

(52) U.S. Cl. CPC A61F 2/2466; A61F 2/2445; A61F 2220/0016; A61F 2220/0075; A61F 2230/0091

(57) ABSTRACT

A method for use with tissue of a heart comprises transluminally advancing a frame into the heart while a wire extends alongside an exterior of the frame. Within the heart, the frame is positioned adjacent the tissue such that the wire becomes positioned against the tissue. While the wire remains positioned against the tissue, a tube is transluminally advanced over and along the wire and alongside the frame to the tissue. While the tube remains positioned alongside the frame, an anchor is advanced along the tube to the tissue. the anchor is subsequently anchored into the tissue. Other embodiments are also described.

The present application is a Continuation of International Patent Application PCT/IB2024/059287 to Pesach et al., filed September 24, 2024, and titled "Systems for guiding and positioning an implant," which published as WO 2025/088396, and which claims priority to Provisional US Patent Application 63/593,935 to Pesach et al., filed on October 27, 2023.

Each of the above documents is incorporated herein by reference in its entirety.

### BACKGROUND

The heart, or portions thereof, may become enlarged under certain conditions. Dilation of an annulus of a heart valve may occur due to various heart conditions, such as an enlarged heart chamber or a leaking heart valve. A heart remodeling or annuloplasty procedure may be necessary to reshape, reinforce or tighten the heart and/or annulus. Annuloplasty may be performed by implanting an annuloplasty implant to re-shape and/or re-size the annulus, for example, to reduce the size of the annulus.

This summary is meant to provide some examples and is not intended to be limiting of the scope of the disclosure in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

The present disclosure relates, inter alia, to methods and systems of implanting an implant along an annulus of a heart of a subject (e.g., a human subject and/or simulation), and for guiding the implantation using various guide assemblies, as described hereinbelow. In some implementations, the implant is adapted to adjust (e.g., reduce) a dimension (e.g., a circumference) of a tissue (e.g., of an annulus) of the heart. For example, the implant may be an annuloplasty implant, configured to reduce regurgitation of a valve (e.g., an atrioventricular valve, such as a tricuspid or mitral valve) of the heart.

The implant can comprise a series of anchors (e.g., sutures, helical anchors, staples, pledget anchors, toggle anchors and/or darts), for anchoring into tissue of a valve annulus (e.g., into tissue of the annulus and/or into tissue of leaflets of the valve). In some implementations, one or more tethers tether the series of anchors to one another, such that anchoring the series of anchors around the tissue positions the tether circumferentially along the valve annulus (e.g., along an atrial surface thereof). The tether(s) can then be subsequently tensioned (e.g., contracted), in order to contract the tissue. For example, this contraction may be used to circumferentially reduce the size of the valve annulus.

In some implementations, a guide assembly, for guiding the implantation of the implant, may include a guide frame that is positionable within a native valve of the heart. While expanded and at the native valve, the guide frame (e.g., a midsection thereof) may press radially against the periphery of the valve, e.g., against tissue of the annulus.

In some implementations, the guide frame may be configured to allow the valve to function at least in part, e.g., by the guide frame being open and allowing blood-flow therethrough, and/or because the leaflets of the valve remain partially functional adjacent the guide frame.

In some implementations, the guide frame defines a plurality of struts, such that, when expanded within the heart, a lattice-like structure defined by the struts is formed.

In some implementations, the guide assembly is a component of a delivery assembly that also includes at least one anchor driver. Such a delivery assembly may be adapted to facilitate implantation of the implant, guided by the guide assembly.

In some implementations, the delivery assembly is adapted to facilitate the anchoring of the anchors circumferentially around the annulus. For example, the guide assembly may predetermine a position and/or orientation at which each of the anchors of the series are to be anchored. Furthermore, the delivery assembly may be configured to prevent the tool that anchors the anchors to the tissue from straying laterally and away from the annulus, by guiding the implantation of the implant around the guide frame.

Systems and methods for guiding the implantation of the series of anchors circumferentially around the annulus are now described.

In some implementations, the guide assembly is a component of a delivery assembly that includes a tool (e.g., a catheter) that is adapted to position and guide the implantation of the implant along the tissue. For example, in some implementations, the tool is an anchor catheter, through which an anchor driver, adapted to anchor the series of anchors to the tissue, is advanced. In some implementations, the tool is adapted to facilitate the anchoring of each anchor of the series sequentially along the valve annulus, such that the implant assumes an arc along the annulus.

In some implementations, during the implantation of the implant along the annulus, the guide assembly utilizes a rack-and-pinion mechanism between the tool and the guide frame, in order to guide the tool around the annulus.

In some implementations, the tool defines a plurality of teeth, adapted to engage the lattice of the guide frame, such that rotation of the tool drives the tool circumferentially around the guide frame, e.g., with the teeth serving as a pinion and the lattice serving as a rack. This advantageously can facilitate controlled advancement of the tool around the annulus and/or stabilization of the tool during anchoring of each of the anchors.

Methods/techniques for guiding an implant circumferentially around the annulus are now further described. In some implementations, a series of guides (e.g., wires) extend, from outside of the subject, transluminally through the delivery assembly to the heart, where the guides extend longitudinally along an exterior of the guide frame, e.g., reaching part of the guide frame that will be positioned downstream of the valve being treated.

In some implementations, the anchors are anchored to the heart by, for each anchor of the series, transluminally sliding the anchor through the delivery assembly (e.g., via a catheter thereof) and into the heart over and along a corresponding guide (e.g., in a zipline-like fashion), until the anchor abuts tissue of the annulus.

In some implementations, once the anchor abuts the tissue of the annulus, the anchor can be anchored to tissue of the annulus. In this manner, the annulus advantageously acts as a flange for the anchor, thereby advantageously desensitizing the system to the position (e.g., depth) within the valve of the guide frame. This may thus obviate the need to ensure that a midsection of the guide frame (e.g., a section around which the anchors will be implanted) is positioned uniformly along the native valve.

In some implementations, a delivery assembly is now described that is adapted to deliver both a guide assembly that includes a guide frame, and an implant that is to be positioned therearound, to the heart. In some implementations, the delivery assembly can be used with the guide assembly that includes the series of guides, as described hereinabove.

In some implementations, the delivery assembly includes a sheath (e.g., a catheter), which is transluminally advanceable through vasculature to the heart of a subject (e.g., a living subject and/or simulation).

In some implementations, the guide frame is advanced to the heart within a primary lumen (e.g., a major lumen region) of the sheath (e.g., in a compressed state), with a control shaft of the guide frame extending, from the guide frame, proximally through the primary lumen and out of the subject (e.g., to provide an extracorporeal control for the guide frame).

In some implementations, once the guide frame is delivered out of the sheath (e.g., out of a distal end thereof) and into the heart, the control shaft can be shifted laterally to a side-lumen (e.g., a minor lumen region) of the sheath, e.g., in order to make space for an anchor driver of the delivery assembly to be advanced through the primary lumen, in order to anchor the series of anchors around the annulus, guided by the guide frame. In some implementations, the control shaft can be narrower than the guide frame.

In some implementations, a series of guides extend longitudinally along an exterior of the guide frame, e.g., reaching part of the guide frame that will be positioned downstream of the valve being treated (e.g., a downstream section thereof). In some implementations, the guides may loop around the guide frame (e.g., around a strut of the downstream section of the guide frame), and then extend proximally back along the guide frame (e.g., in a similarly longitudinal fashion), into the delivery assembly.

In some implementations, the sheath may define multiple secondary lumens to house the guides, such that during delivery of the guide frame to the heart, each guide may be concealed within an individual secondary lumen of the catheter. For example, each guide can extend, from a first end of the guide that is outside of the subject, through a secondary lumen, where the guide loops around a downstream section of the guide frame, and then back through that secondary lumen to a second end that is disposed out of the subject. In this manner, the sheath may advantageously prevent the guides from becoming tangled and/or in the passage of the anchors which are to be subsequently anchored to the tissue via the sheath.

In some implementations, each of the secondary lumens of the catheter may be connected, along its length, to the primary lumen via an elongate slit. Once the guide frame is positioned within the heart (e.g., and the control shaft of the guide frame has been shifted to the side-lumen), an anchor tool (e.g., comprising an anchor driver and/or a catheter that is adapted to house an anchor driver) can be delivered through the primary lumen, engaged with an anchor of the implant.

In some implementations, the anchor tool may be threaded onto a guide of the series, such that advancement of the anchor tool involves sliding the anchor tool over and along the guide, towards the heart.

In some implementations, as the anchor catheter is advanced progressively distally through the primary lumen, it pulls progressively distal portions of the guide through progressively distal portions of the corresponding slit and into the primary lumen. Within the heart further sliding of the anchor tool over and along the guide brings the anchor tool to tissue of the annulus (e.g., such that the anchor engaged by the anchor tool abuts the annulus), such that the anchor can be anchored to the tissue (e.g., using the anchor driver). In this manner, the guides may advantageously define the position and/or spacing along the annulus at which the anchors are anchored.

In some implementations, once the anchor has been anchored to the annulus, the anchor tool can be retracted (e.g., along with the corresponding guide, which may be unlooped from the guide frame), leaving the anchor anchored to the tissue. This process can be repeated for the other anchors of the series.

In some implementations, a tether extends along the anchors, such that once the anchors have been implanted along the annulus, the tether can be contracted to reshape (e.g., to contract) the annulus.

In some implementations, a guide frame is used that has a series of flaps attached around the midsection of the guide frame, each flap having a guide (e.g., a variant of the guides described hereinabove) that extends, from the flap, proximally through the delivery assembly, and out of the subject. In some implementations, the guide frame can be delivered to the heart with the flaps held in a closed state against the guide frame, for example, by maintaining tension on each guide, such as by pulling the guides proximally.

In some implementations, upon positioning of the guide frame at the native valve (e.g., such that the midsection abuts tissue of the annulus), the flaps can be opened by actuating the guides (e.g., by pushing distally on the guides and/or by releasing tension on the guides), such that each flap extends outwardly from the guide frame, and away from the valve orifice, against the surface of the annulus. In some implementations, opening the flaps causes the guide assembly to assume a flower-like appearance on the annulus, e.g., by each flap (or “petal”) extending radially outwards from the guide frame, on the surface of the annulus.

In order to implant the series of anchors around the annulus, each anchor of the series can then be delivered to the heart over and along a corresponding guide (e.g., similarly to the “zipline” technique described hereinabove), until the anchor reaches its corresponding flap. In some implementations, the anchor can then be anchored to the tissue, e.g., by sandwiching the flap between the anchor (e.g., a head thereof), and the tissue. This technique may advantageously allow, for each anchor of the series, for the distance between the anchor and the valve orifice to be predefined (e.g., preselected), e.g., such that all anchors are anchored uniformly at a predetermined distance from the valve orifice.

In some implementations, the flaps are intracardially disengageable from the guide frame, to allow for withdrawal of the guide frame from the heart, such that once implantation is complete, the toggle anchors (and the flaps thereof) remain anchored to the tissue.

In some implementations, a tether extends along the anchors, such that once the anchors have been implanted along the annulus, the tether can be contracted to reshape (e.g., contract) the annulus.

In some implementations, the anchor is anchored by driving part of the anchor through the tissue of the annulus such that it becomes exposed within the ventricle. In some implementations, a wire arrangement may be temporarily positioned within the ventricle, to bias the leaflets of the valve medially (e.g., inwardly) away from ventricle wall. For example, this inward biasing of the leaflets may keep the leaflets clear during anchoring of such annulus-traversing anchors.

In accordance with some implementations, a system and/or apparatus usable and/or for use with tissue of a cardiovascular system of a subject is disclosed, the system including a delivery assembly that includes a guide frame and/or a tool.

In some implementations, the delivery assembly is configured to position the guide frame adjacent the tissue.

In some implementations, the tool has radial teeth adapted to engage the guide frame such that rotation of the tool drives the tool along the guide frame. The tool may be configured to guide a treatment of the tissue via the driving of the tool along the guide frame.

In some implementations, the guide frame has an exterior surface and an interior surface. The delivery assembly may be configured to position the guide frame such that the exterior surface faces the tissue.

In some implementations, the tool may be configured such that the rotation of the tool drives the tool along the interior surface of the guide frame. In some implementations, the tool may be configured such that the rotation of the tool drives the tool along the exterior surface of the guide frame.

In some implementations, the delivery assembly includes a sheath.

In some implementations, the sheath defines a major lumen region, and/or a minor lumen region.

In some implementations, the minor lumen region has a smaller cross-sectional area than the major lumen region.

In some implementations, the delivery assembly may be configured to transluminally advance the guide frame into the cardiovascular system while the guide frame is disposed within the major lumen region of the sheath, and/or a control shaft coupled to the guide frame extends, from the guide frame, proximally through the major lumen region.

In some implementations, while the guide frame remains distally outside of the sheath and the control shaft remains coupled to the guide frame, the control shaft may be shiftable laterally out of the major lumen region and into the minor lumen region, such that the tool is advanceable through the major lumen region to the guide frame.

In some implementations, the guide frame is intracorporeally expandable at the tissue.

In some implementations, the tool includes a catheter, configured to guide the treatment of the tissue by guiding a treatment assembly that is advanceable through the catheter.

In some implementations, the delivery assembly is configured to bias the tool against the guide frame.

In some implementations, the system further includes a sheath, and the system is configured to extend both the tool and the guide frame distally out of the sheath at the tissue in a manner that biases the tool against the guide frame.

In some implementations, the teeth are intracorporeally transitionable into a protruding state in which the teeth protrude radially outward from a surface of the tool.

In some implementations, the tool is advanceable toward the tissue while the teeth are in a retracted state in which the teeth lie flush with the surface of the tool.

In some implementations, the guide frame comprises struts that define windows therebetween, and the teeth are adapted to engage the guide frame by the teeth protruding into the windows of the guide frame.

In some implementations, the struts are arranged in a lattice-like framework.

In some implementations, the system further includes an implant, and the tool is configured to guide the treatment of the tissue by guiding the implantation of the implant at the tissue.

In some implementations, the implant is an elongate implant, and the tool is configured to guide the implantation of the implant alongside the guide frame.

In some implementations, the implant is an elongate implant, and the tool is configured to guide the implantation of the implant along the tissue.

In some implementations, the tissue is tissue of an annulus of a heart of the subject, the annulus circumscribing an orifice of the valve.

In some implementations, the elongate implant is an annuloplasty implant including a series of anchors.

In some implementations, the delivery assembly may be configured to position the guide frame adjacent the tissue by positioning the guide frame through the orifice such that the guide frame is adjacent the tissue of the annulus.

In some implementations, the tool may be configured to guide the treatment of the tissue by guiding the implantation of the anchors around the annulus, such that the implant extends along the annulus.

In some implementations, the anchors of the series are connected via a tether, and the tool is configured to guide the treatment of the tissue by guiding the implantation of the anchors around the annulus, such that the tether extends along the annulus.

In some implementations, the delivery assembly is configured to tension the tether to reduce a circumference of the annulus.

In some implementations, the tool comprises a catheter, transluminally advanceable to the heart, and the system further includes an anchor driver, adapted to, one or more anchors of the series, engage the anchor and to deliver the anchor to the heart, via the catheter.

In accordance with some implementations, a method usable and/or for use of a system and/or apparatus at a tissue of a subject (e.g., a living subject or simulation) includes advancing, into the subject a guide fame and/or a tool.

In some implementations, the tool may be configured to deliver a treatment to the tissue. In some implementations, the tool may have radial teeth.

In some implementations, the method may include positioning the guide frame adjacent the tissue, and/or driving the tool along the guide frame by rotating the tool while the radial teeth are engaged with the guide frame.

In some implementations, the method further includes delivering a treatment along the tissue, facilitated by the driving of the tool along the guide frame.

In some implementations, delivering the treatment along the tissue includes delivering the treatment out of the tool and along the tissue.

In some implementations, the tissue is tissue of an annulus of a valve of a heart of the subject, and delivering the treatment along the tissue includes contracting a circumference of the annulus.

In some implementations, the method further includes intracorporeally expanding the guide frame, subsequent to advancing the guide frame into the subject.

In some implementations, advancing the guide frame into the subject includes transluminally advancing the guide frame into the subject while the guide frame is constrained within a compressed state.

In some implementations, the method further includes, subsequently to advancing the tool into the subject, intracorporeally transitioning the teeth into a protruding state in which the teeth protrude radially outward from a surface of the tool.

In some implementations, advancing the tool into the subject includes transluminally advancing the tool into the subject while the teeth are in a retracted state in which the teeth lie flush with the surface of the tool.

In some implementations the guide frame comprises struts that define windows therebetween. Driving the tool along the guide frame by rotating the tool while the radial teeth are engaged with the guide frame may include driving the tool along the guide frame by rotating the tool while the radial teeth protrude into the windows of the guide frame.

In some implementations, the struts are arranged in a lattice-like framework, and driving the tool along the guide frame includes driving the tool along the lattice-like framework of the guide frame.

In some implementations, the method further includes implanting an implant at the tissue, guided by the driving of the tool along the guide frame.

In some implementations, the implant is an elongate implant, and implanting the implant at the tissue includes implanting the elongate implant at the tissue.

In some implementations, the tissue is tissue of an annulus of a heart of the subject, the annulus circumscribing an orifice of the valve.

In some implementations, the elongate implant may be an annuloplasty implant including a series of anchors.

In some implementations, positioning the guide frame adjacent the tissue includes positioning the guide frame through the orifice. In some implementations, implanting the elongate implant at the tissue may include implanting the annuloplasty implant at the tissue, by driving the tool around the guide frame, and intermittently pausing the driving to anchor an anchor of the series to the tissue.

In some implementations, implanting the annuloplasty implant at the tissue includes, for one or more anchors of the series, engaging the anchor with an anchor driver, and delivering, via the tool, the anchor driver to the heart, and subsequently, using the anchor tool to anchor the anchor to the tissue.

In some implementations, the anchors of the series are connected via a tether, and implanting the implant along the tissue of the annulus includes implanting the series of anchors along the tissue such that the tether extends along the annulus.

In some implementations, the method further includes tensioning the tether to reduce a circumference of the annulus.

In some implementations, tensioning the tether to reduce the circumference of the annulus includes tensioning the tether to reduce the circumference of the annulus by contracting the guide frame radially inward while the guide frame is coupled to one or more anchors of the series.

In accordance with some implementations, a system and/or apparatus (which can be usable and/or for use with tissue of a heart of a living subject or simulation) is disclosed, the system including an implant, a delivery assembly, and/or an anchor tool.

In some implementations, the implant may include a series of anchors.

In some implementations, the delivery assembly can include a guide assembly that includes a guide frame and/or multiple guides. One or more of the multiple guides may extend longitudinally alongside an exterior of the guide frame to a respective coupling point.

In some implementations, the delivery assembly may be configured to transluminally advance the guide frame into the heart. In some implementations, the delivery assembly may be configured to within the heart, position the guide frame adjacent the tissue.

In some implementations, the delivery assembly may be configured to implant the implant while the guide frame remains positioned adjacent the tissue by, for one or more anchors of the series: (i) transluminally sliding the anchor tool over and along a guide of the multiple guides, and alongside the guide frame, to an anchor site at the tissue adjacent the guide frame, and/or (ii) using the anchor tool, anchoring the anchor to the tissue at the anchor site. The delivery assembly may be further configured to subsequently withdraw the guide frame from the heart.

In some implementations, the delivery assembly includes a sheath that comprises a major lumen region, and/or a minor lumen region. In some implementations, the minor lumen region may have a smaller cross-sectional area than the major lumen region.

In some implementations, the delivery assembly may be configured to transluminally advance the guide frame into the heart while the guide frame is disposed within the major lumen region of the sheath. In some implementations, a control shaft coupled to the guide frame may extend, from the guide frame, proximally through the major lumen region.

In some implementations, while the guide frame remains distally outside of the sheath and the control shaft remains coupled to the guide frame, the control shaft is shiftable laterally out of the major lumen region and into the minor lumen region, such that the anchor tool is advanceable through the major lumen region to the anchor site.

In some implementations, one or more anchors of the series is a helical anchor, and the anchor tool is adapted to anchor the anchor to the tissue by screwing the helical anchor into the tissue.

In some implementations, one or more anchors of the series is a pledget anchor.

In some implementations, one or more of the multiple guides is a wire.

In some implementations, one or more of the multiple guides is a suture.

In some implementations, the guide frame defines a framework of struts, such that one or more guides of the multiple guides loop around a respective strut of the guide frame at its respective coupling point.

In some implementations, the one or more guides extends, from a first end that is outside of the heart, to the heart, where the guide loops around the guide frame at the coupling point, and back out of the heart. In some implementations, the one or more guides are intracardially decouplable from the guide frame by pulling an end of the guide proximally.

In some implementations, the anchor tool comprises a lumen. In some implementations, the delivery assembly may be configured to slide the anchor tool over and along one of the guides by sliding the lumen over and along the guide.

In some implementations, the lumen is an oblique lumen at a distal end portion of the anchor tool. In some implementations, the tool may have an intermediate portion proximal from the distal end portion.

In some implementations, the delivery assembly may be configured to slide the lumen over and along the guide by sliding the oblique lumen distally along the guide while the intermediate portion is disposed alongside the guide.

In some implementations, the guide extends, from an extracorporeal end of the guide, transluminally through the delivery assembly and longitudinally alongside an exterior of the guide frame to a coupling point.

In some implementations, the delivery assembly includes a sheath that comprises a primary lumen, and multiple secondary lumens disposed alongside the primary lumen. In some implementations, the sheath may define multiple slits, such that a slit connects a respective secondary lumen to the primary lumen. In some implementations, the guides may be housed within a corresponding secondary lumen.

In some implementations, the delivery assembly may be configured to slide the anchor tool distally through the primary lumen such that the anchor tool pulls progressively distal portions of the respective guide laterally through the corresponding slit and into the primary lumen for the one or more anchors.

In some implementations, the multiple secondary lumens are arranged circumferentially around the primary lumen.

In some implementations the anchor tool includes an anchor catheter, and/or an anchor driver. In some implementations, the anchor driver may be advanceable through a lumen of the anchor catheter. In some implementations, the delivery assembly may be configured to slide the anchor tool over and along a guide by sliding the anchor catheter over and along the guide. In some implementations, the delivery assembly may be configured to anchor the anchor to the tissue at the anchor site using the anchor driver while the anchor driver extends through the lumen of the anchor catheter.

In some implementations, the lumen of the anchor catheter is a driver lumen and is eccentric. In some implementations, the anchor catheter may further comprise a guide lumen that is eccentric. In some implementations, the delivery assembly may be configured to slide the anchor catheter over and along one of the guides by sliding the guide lumen over and along the guide.

In some implementations, the delivery assembly is configured to position the guide frame adjacent the tissue by expanding the guide frame within the heart.

In some implementations, the delivery assembly is configured to position the guide frame adjacent the tissue by expanding the guide frame within the heart until the guide frame abuts the tissue.

In some implementations, the delivery assembly is configured to position the guide frame adjacent the tissue such that the coupling point of the guide is distal to the tissue. In some implementations, the delivery assembly may be configured to, for the anchor, slide the anchor tool distally over and along the guide until the anchor tool abuts the tissue at a respective anchoring site, proximal from the coupling point.

In some implementations, the delivery assembly is configured to sandwich at least one of the guides between the guide frame and the tissue, such that the tissue obstructs the anchor tool from reaching the coupling point.

In some implementations, the tissue is tissue of an annulus of a valve of the heart, the annulus circumscribing an orifice of the valve. In some implementations, the implant may be an annuloplasty implant including the series of anchors. In some implementations, the delivery assembly may be configured to position the guide frame through the orifice such that the multiple guides extend through the orifice alongside the exterior of the guide frame. For one or more anchors of the series, the delivery assembly may be configured to slide the tool alongside the guide frame until the tool abuts the annulus.

In some implementations, the implant is an elongate implant, and the delivery assembly is configured to guide the implantation of the elongate implant circumferentially around the guide frame by anchoring the one or more anchors of the series of anchors to the respective anchor sites.

In some implementations, the tissue is tissue of an annulus of an atrioventricular valve of the heart. In some implementations, the valve may have an atrium upstream of the valve, and a ventricle downstream of the valve. In some implementations, the elongate implant may be an annuloplasty implant that includes the series of anchors. In some implementations, the delivery assembly may be configured to guide the implantation of the elongate implant circumferentially around the guide frame such that the implant extends along the annulus.

In some implementations, the one or more anchors are toggle anchors. In some implementations, the delivery assembly may be configured to anchor the one or more anchors of the series by advancing the anchor, from the atrium through the tissue of the annulus and into the ventricle.

In some implementations, the implant further includes a tether that connects the anchors of the series. In some implementations, the tool is configured to implant the implant such that the tether extends along the annulus.

In some implementations, the delivery assembly is configured to reduce a circumference of the annulus by contracting the guide frame while the anchors remain anchored to the annulus.

In some implementations, the delivery assembly is configured to reduce a dimension of the annulus by applying tension to the tether while the anchors remain anchored to the annulus.

In some implementations, the delivery assembly is configured to maintain the annulus at the reduced dimension by locking the tension in the tether.

In some implementations, the guide assembly further includes multiple flaps spaced circumferentially along the exterior of the guide frame. In some implementations, a guide of the multiple guides may be coupled to the guide frame via a respective one of the flaps. In some implementations, the flaps may be intracorporeally deflectable away from the guide frame in a manner that spaces the respective guide from the guide frame.

In some implementations, for an anchor, the delivery assembly is configured to anchor the anchor to the tissue at the anchor site by delivering the anchor through a flap and into the tissue.

In some implementations, a flap of the multiple flaps is deflectable from a retracted position in which the flap lies against the exterior of the guide frame, towards a deployed position in which the flap lies against the tissue.

In some implementations, the flap is transitionable to the deployed position by pushing its corresponding guide distally.

In some implementations, the flap is attached to the guide frame via a connecting wire that weaves in and out of the guide frame. At the flap, the connecting wire exposed out of the guide frame and loops through the flap.

In some implementations, the flap is intracardially disengageable from the guide frame by retracting the connecting wire, such that the connecting wire unloops from the flap.

In some implementations, the delivery assembly is configured to, an anchor of the series of anchors, anchor the anchor to the tissue in a manner that couples the anchor to a corresponding flap.

In some implementations, the delivery assembly may be configured to retract the connecting wire to disengage the guide frame from the flap. In some implementations, the delivery assembly may be configured to withdraw the guide frame from the heart such that the flap remains coupled to a corresponding anchor within the heart.

In some implementations, the anchors are toggle anchors. In some implementations, the toggle anchor may have a toggle, and a cord that extends away from the toggle. In some implementations, the delivery assembly may be configured to anchor an anchor of the series by advancing the toggle, from a first chamber of the heart, through the flap and the tissue, into a second chamber of the heart, such that the cord extends from the toggle in the second chamber, through the tissue and the flap, to an anchor head that is disposed against the flap within the first chamber.

In some implementations, the system may be configured to retract the connecting wire to disengage the guide frame from the flaps. In some implementations, the system may be configured to withdraw the guide frame from the heart such that each flap comprises a first component of the toggle anchor, and each anchor comprises a second component of the toggle anchor.

In accordance with some implementations, a method usable and/or for use of a system and/or apparatus with tissue of a heart (e.g., of a living subject or of a simulation) includes transluminally advancing a guide frame into the heart while multiple guides extend longitudinally alongside an exterior of the guide frame to respective coupling points on the guide frame.

In some implementations, the method may include, within the heart, positioning the guide frame adjacent the tissue. In some implementations, the method may further include, while the guide frame remains adjacent the tissue, sliding an anchor tool over and along a guide of the multiple guides, and alongside the guide frame, to a site at the tissue that is adjacent the guide frame.

In some implementations, the method may further include, subsequently, using the anchor tool, anchoring an anchor to the tissue at the anchor site, and/or subsequently, withdrawing the guide frame from the heart.

In some implementations, the anchor of the series of anchors is a helical anchor, and anchoring the anchor to the tissue includes screwing the helical anchor into the tissue.

In some implementations, the anchor of the series of anchors is a pledget anchor, and anchoring the anchor to the tissue includes anchoring the pledget anchor to the tissue.

In some implementations, a guide of the multiple guides is a wire.

In some implementations, a guide of the multiple guides is a suture.

In some implementations, the guide frame comprises a framework of struts, the guide looping around a respective strut of the guide frame at a coupling point, and positioning the guide frame adjacent the tissue includes positioning the guide frame adjacent the tissue, such that the coupling point is positioned distally to the tissue.

In some implementations, each guide extends, from a first end that is outside of the heart, to the heart, where the guide loops around the guide frame at the coupling point, and back out of the heart. The method may further include intracardially decoupling each guide from the guide frame by pulling an end of the guide proximally.

In some implementations, transluminally advancing the guide frame into the heart includes transluminally advancing the guide frame into the heart via a major lumen region of a sheath, with a control shaft coupled to the guide frame that extends, from the guide frame, proximally through the major lumen region. In some implementations, positioning the guide frame adjacent the tissue may include delivering the guide frame distally outside of the sheath and positioning the guide frame adjacent the tissue.

In some implementations, while the guide frame remains distally out of the sheath, and the control shaft remains coupled to the guide frame, the method may further include shifting the control shaft laterally out of the major lumen region and into a minor lumen region of the sheath. In some implementations, the minor lumen region may have a smaller cross-sectional area than the major lumen region. In some implementations, sliding the anchor tool over and along the guide may include sliding the anchor tool through the major lumen region, over and along the guide.

In some implementations, shifting the control shaft laterally out of the major lumen region and into the minor lumen region includes shifting the control shaft laterally out of the major lumen region and into the minor lumen region by sliding the anchor tool through the major lumen region, such that the control shaft is pushed out of the major lumen region and into the minor lumen region.

In some implementations, the anchor tool comprises a lumen. In some implementations, sliding the anchor tool over and along the respective one of the guides may include sliding the lumen over and along the respective one of the guides.

In some implementations, the lumen is an oblique lumen at a distal end portion of the anchor tool, the tool having an intermediate portion proximal from the distal end portion. In some implementations, sliding the lumen over and along the guide may include sliding the oblique lumen distally over and along the guide while the intermediate portion is disposed alongside the guide.

In some implementations, sliding the anchor tool over and along the guide includes sliding the anchor tool over and along the guide while the anchor tool is housed within a sheath. In some implementations, the sheath may define a primary lumen, multiple secondary lumens disposed alongside the primary lumen, the guide being housed within a corresponding secondary lumen, and/or multiple slits, a slit of the multiple slits connecting the corresponding secondary lumen to the primary lumen.

In some implementations, sliding the anchor tool over and along the guide may include sliding the anchor tool distally within the primary lumen, such that the anchor tool pulls progressively distal portions of the guide laterally through the corresponding slit and into the primary lumen.

In some implementations, the multiple secondary lumens are arranged circumferentially around the primary lumen.

In some implementations, the anchor tool includes an anchor catheter, and/or an anchor driver extendable through a lumen of the anchor catheter. In some implementations, sliding the anchor tool over and along the guide may include sliding the anchor catheter over and along a guide. In some implementations, anchoring the anchor to the tissue may include using the anchor driver to anchor the anchor to the tissue, while the anchor driver is extended within a lumen of the anchor catheter.

In some implementations, the lumen of the anchor catheter is a driver lumen and is eccentric. In some implementations, the anchor catheter may further comprise a guide lumen that is eccentric. In some implementations, sliding the anchor tool over and along the guide may include sliding the guide lumen over and along the guide.

In some implementations, positioning the guide frame adjacent the tissue includes expanding the guide frame within the heart until the guide frame abuts the tissue.

In some implementations, expanding the guide frame within the heart until the guide frame abuts the tissue includes expanding the guide frame within the heart until the guide frame presses against the tissue.

In some implementations, positioning the guide frame adjacent the tissue includes positioning the guide frame adjacent the tissue such that the respective coupling points are positioned distally to the tissue. In some implementations, sliding the anchor tool over and along guide may include sliding the anchor tool over and along the guide until the anchor tool abuts the tissue at a respective anchoring site, the anchoring site being proximal from the respective coupling point.

In some implementations, positioning the guide frame adjacent the tissue includes positioning the guide frame adjacent the tissue in a manner that sandwiches the guide between the guide frame and the tissue, such that the tissue obstructs the anchor tool from reaching the coupling point.

In some implementations, the tissue is tissue of an annulus of a valve of the heart, the annulus circumscribing an orifice of the valve. In some implementations, the anchor may be an anchor of an annuloplasty implant.

In some implementations, positioning the guide frame adjacent the tissue may include positioning the guide frame through the orifice such that the guide extends through the orifice alongside the exterior of the guide frame. In some implementations, sliding the anchor tool over and along the guide may include sliding the anchor tool over and along the guide until the anchor tool abuts the annulus.

In some implementations, the anchor is an anchor of a series of anchors of an elongate implant. In some implementations, the method may further include guiding the implantation of the elongate implant circumferentially around the guide frame by, for each anchor of the series of anchors: (i) sliding the anchor tool over and along a respective one of the guides, and alongside the guide frame, to a respective site at the tissue that is adjacent the guide frame, and/or (ii) anchoring the anchor to the anchor site along the tissue.

In some implementations, the tissue is tissue of an annulus of an atrioventricular valve of the heart, the valve having an atrium upstream of the valve, and a ventricle downstream of the valve. In some implementations, the elongate implant may be an annuloplasty implant including the series of anchors.

In some implementations, guiding the implantation of the elongate implant circumferentially around the guide frame may include guiding the implantation of the annuloplasty implant circumferentially around the guide frame such that the annuloplasty implant extends along the annulus.

In some implementations, the anchor is a toggle anchor. Anchoring the anchors of the series to respective anchor sites along the tissue may include, for each anchor of the series, advancing the anchor, from the atrium through the tissue of the annulus and into the ventricle.

In some implementations, the annuloplasty implant further includes a tether that connects the anchors of the series, and guiding the implantation of the annuloplasty implant circumferentially around the guide frame includes guiding the implantation of the annuloplasty implant circumferentially around the guide frame such that the tether extends along the annulus.

In some implementations, the method further includes reducing a dimension of the annulus by applying tension to the tether while the anchors remain anchored to the annulus.

In some implementations, the method further includes maintaining the annulus at the reduced dimension by locking the tension in the tether.

In some implementations, applying tension to the tether includes applying tension to the tether by contracting the guide frame while the anchors remain anchored to the annulus.

In some implementations, advancing the guide frame into the heart includes advancing the guide frame into the heart while multiple flaps are spaced circumferentially along the exterior of the guide frame. In some implementations, the guide may be coupled to the guide frame via a flap of the multiple flaps.

In some implementations, the method may include, subsequently to positioning the guide frame adjacent the tissue, and prior to sliding the anchor tool over and the guide, deflecting the flap away from the guide frame in a manner that spaces the guide away from the guide frame.

In some implementations, anchoring the anchor to the tissue includes anchoring the anchor adjacent the flap and into the tissue.

In some implementations, the method further includes, subsequent to anchoring the anchor adjacent the flap, withdrawing the flap from the heart.

In some implementations, deflecting the flap away from the guide frame includes deflecting the flap, from a retracted position in which the flap lies against the exterior of the guide frame, towards a deployed position in which the flaps lie against the tissue.

In some implementations, deflecting the flap away from the guide frame includes pushing the guide distally.

In some implementations, the flaps are attached to the guide frame via a connecting wire that: (i) weaves in and out of the guide frame, and/or (ii) at a flap of the multiple flaps, is exposed out of the guide frame and loops through the flap. In some implementations, sliding the anchor tool over and along the respective one of the guides may include sliding the anchor tool over and along the respective one of the guides while the flap is connected to the guide frame via the connecting wire.

In some implementations, the method further includes intracardially disengaging the flaps from the guide frame by retracting the connecting wire, such that the connecting wire unloops from the multiple flaps.

In some implementations, anchoring the anchor to the tissue includes delivering the anchor through the flap and into the tissue.

In some implementations, anchoring the anchor to the tissue includes anchoring the anchor to the tissue in a manner that couples the anchor to the flap.

In some implementations, the method may further include, subsequently to disengaging the flaps from the guide frame, withdrawing the guide frame from the heart such that the flap remains coupled to the anchor within the heart.

In some implementations, the anchor comprises a toggle anchor having a toggle, and a cord that extends away from the toggle. In some implementations, anchoring the anchor to the tissue may include advancing the toggle, from a first chamber of the heart, through the flap and the tissue, into a second chamber of the heart, such that the cord extends from the toggle in the second chamber, through the tissue, to the flap in the first chamber. Withdrawing the guide frame from the heart may include withdrawing the guide frame from the heart such that the flap comprises a first component of the toggle anchor, and the anchor comprises a second component of the toggle anchor.

In accordance with some implementations, a method for using a system or apparatus with a heart of a subject (e.g., a living subject or simulation) is disclosed, the method including transluminally advancing a distal end of a sheath toward the heart. In some implementations, the sheath may comprise a lumen therethrough, the lumen having, at least at the distal end, a major lumen region, and/or a minor lumen region. The minor lumen region may have a smaller cross-sectional area than the major lumen region.

In some implementations, the method may further include positioning the distal end of the sheath within the heart while a control shaft extends distally through the major lumen region. In some implementations, the method may further include using the control shaft, pushing a device, coupled to a distal end of the control shaft, distally out of the major lumen region to become disposed distally outside of the sheath. In some implementations, the method may further include, while the device remains distally outside of the sheath and coupled to the control shaft, shifting the control shaft laterally out of the major lumen region and into the minor lumen region.

In some implementations, the method further includes, subsequent to pushing the device distally out of the sheath, expanding the device within the heart.

In some implementations, a cross-sectional area of the device is larger than the cross-sectional area of the minor lumen region.

In some implementations, the device is a first device, and/or the method further includes, while the first device remains distally outside of the sheath, advancing a second device through the major lumen region and distally out of the sheath.

In some implementations, shifting the control shaft laterally out of the major lumen region and into the minor lumen region includes advancing the second device through the major lumen region such that the second device progressively pushes the control shaft out of the major lumen region and into the minor lumen region.

In some implementations, advancing the second device through the major lumen region and distally out of the sheath includes advancing the second device through the major lumen region and distally out of the sheath while the second device is coupled to the first device.

In some implementations, the first device is a guide frame. Advancing the second device distally out of the sheath may include advancing the second device distally out of the sheath and towards a site at the heart, guided by the guide frame.

In some implementations, the second device is an anchor tool. In some implementations, the method may further include the method may further include, while the guide frame remains distally outside of the sheath and the control shaft remains in the minor lumen region, anchoring an anchor to the tissue, by: (i) advancing the anchor tool through the major lumen region, while the anchor tool is engaged with the anchor, towards tissue of the heart, and/or (ii) using the anchor tool to anchor the anchor to the tissue.

In accordance with some implementations, an apparatus and/or system usable and/or for use with a first device and a second device is disclosed, the apparatus including a sheath. In some implementations, the method may further include the sheath may be for advancing into vasculature of a subject (e.g., a living subject and/or simulation). In some implementations, the sheath may comprise a lumen therethrough, the lumen having, at least at a distal end of the sheath a major lumen region, and/or a minor lumen region. The minor lumen region may have a smaller cross-sectional area than the major lumen region.

In some implementations, the first device may be transluminally advanceable through the sheath while the first device is disposed within the major lumen region of the sheath and a control shaft extends, from the first device, proximally away from the first device through the major lumen region. In some implementations, the method may further include the first device may be deliverable out of the distal end of the sheath, such that the control shaft extends from the first device, proximally through the major lumen region.

In some implementations, the method may further include the second device may be transluminally advanceable through the major lumen region while the first device is disposed out of the distal end of the sheath and the control shaft remains coupled to the first device, by shifting the control shaft laterally out of the major lumen region and into the minor lumen region to vacate the major lumen region for the second device.

In accordance with some implementations, a method usable and or for use of a system and/or apparatus with tissue of a heart (e.g., heart of a living subject or simulation) includes transluminally advancing a guide frame into the heart while a guide extends longitudinally alongside an exterior of the guide frame to a coupling point on the guide frame.

In some implementations, the method may further include the method may include, within the heart, positioning the guide frame adjacent the tissue such that the coupling point is distally beyond the tissue, and the guide is sandwiched between the guide frame and the tissue. In some implementations, the method may further include the method may further include, while the guide remains sandwiched between the guide frame and the tissue, positioning an implant at the tissue by advancing the implant along the guide alongside the guide frame until the implant abuts the tissue.

In accordance with some implementations, a system for use with tissue of a heart (e.g., a heart of a living subject or simulation) can comprise one or more of an implant comprising a series of anchors, a guide assembly, and/or an anchor tool. In some implementations, the guide assembly can comprise a guide frame and multiple guides, where a guide of the multiple guides extending alongside an exterior of the guide frame to a coupling point.

In some implementations, the guide assembly is configured to be transluminally advanced into the heart and be positioned adjacent the tissue. In some implementation, while the guide frame is positioned adjacent the tissue: the anchor tool may be transluminally slid over and along the guide, and alongside the guide frame, to an anchor site at the tissue adjacent the guide frame, and using the anchor tool, one or more anchors of the series of anchors can be secured at the anchor site.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

### BRIEF DESCRIPTION OF THE DRAWINGS

FIGS. 1A-G illustrate a delivery assembly and its aesthetic features adapted to facilitate the implantation of implants circumferentially around a tissue, in accordance with some implementations,

FIGS. 2A-G illustrate a delivery assembly and its aesthetic features adapted to facilitate the implantation of implants circumferentially around a tissue, in accordance with some implementations, and

FIGS. 3A-G illustrate a delivery assembly and its aesthetic features adapted to facilitate the implantation of implants circumferentially around a tissue, in accordance with some implementations.

### DETAILED DESCRIPTION OF IMPLEMENTATIONS

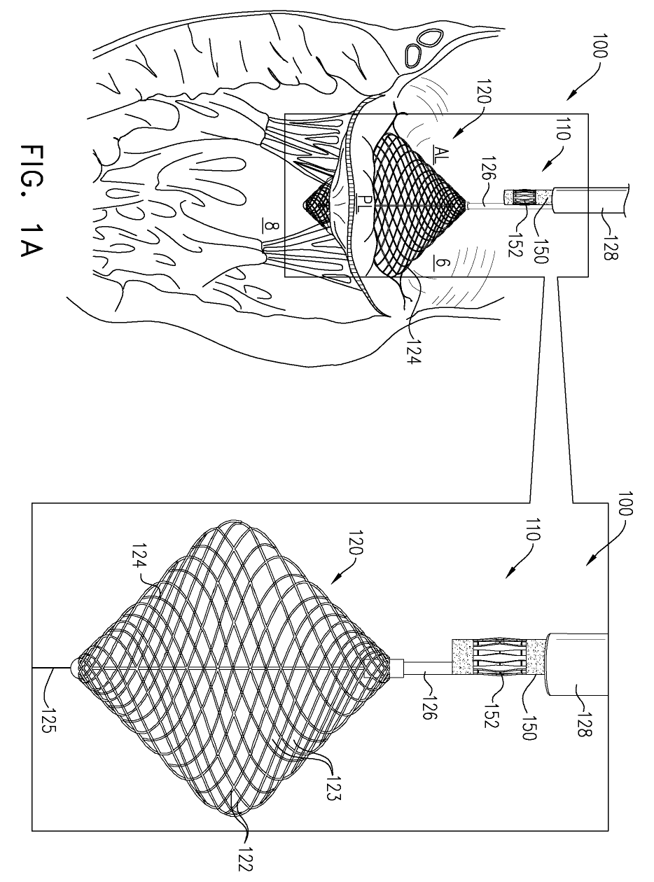

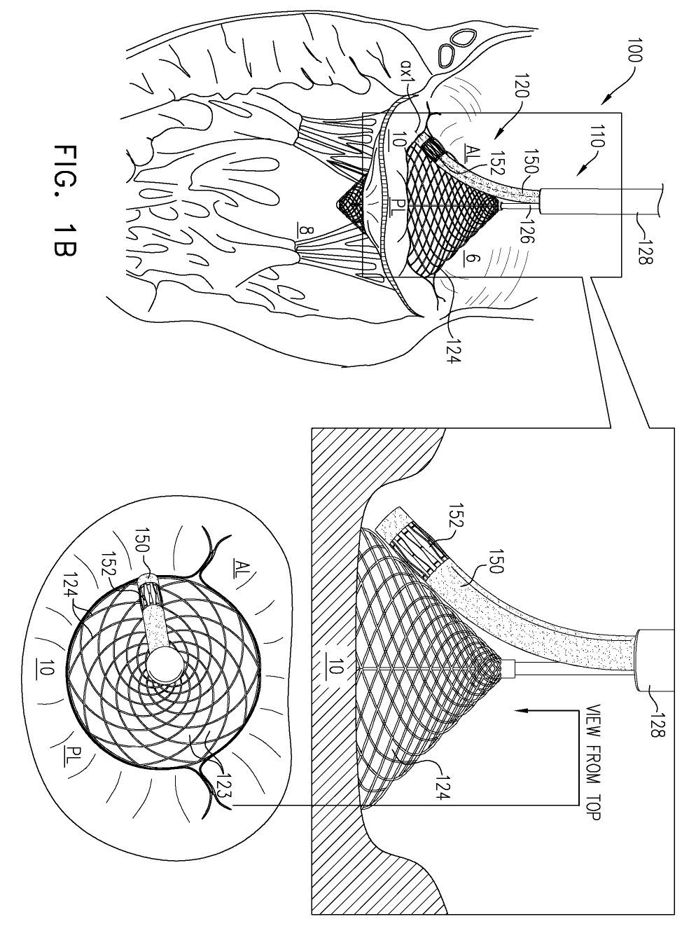

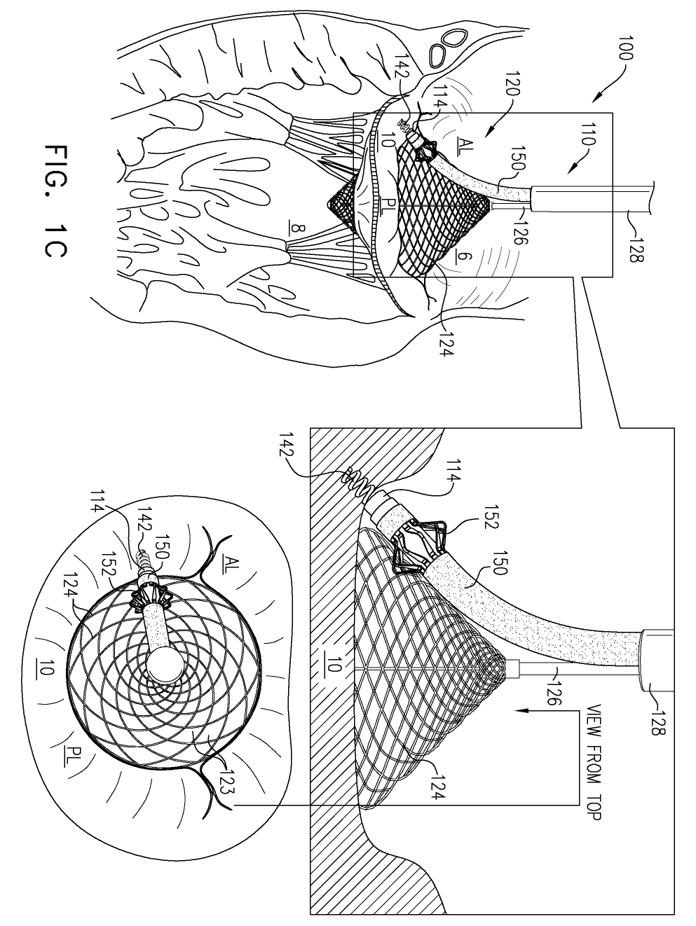

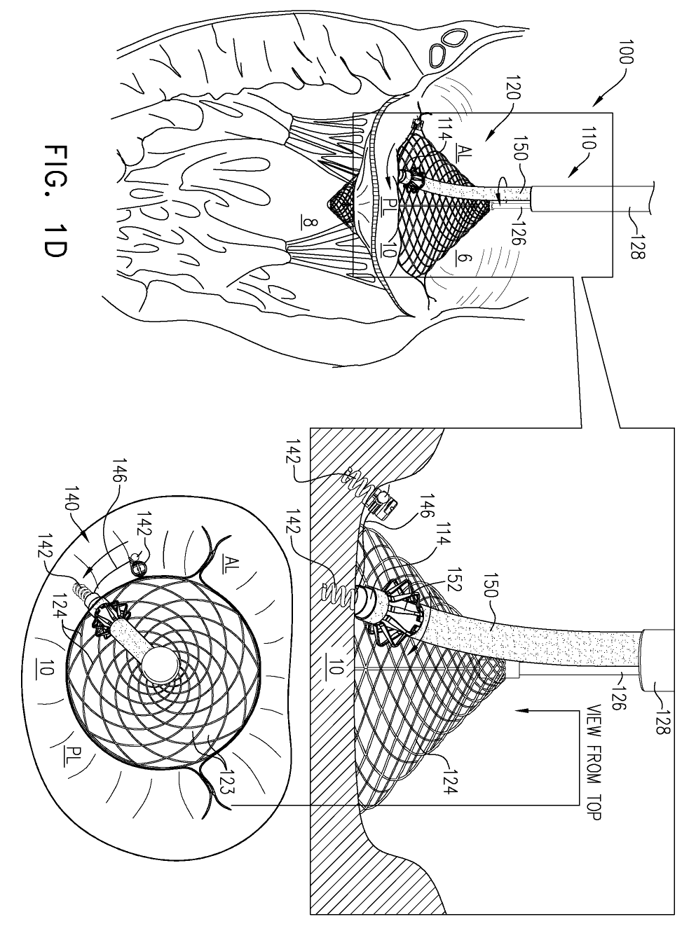

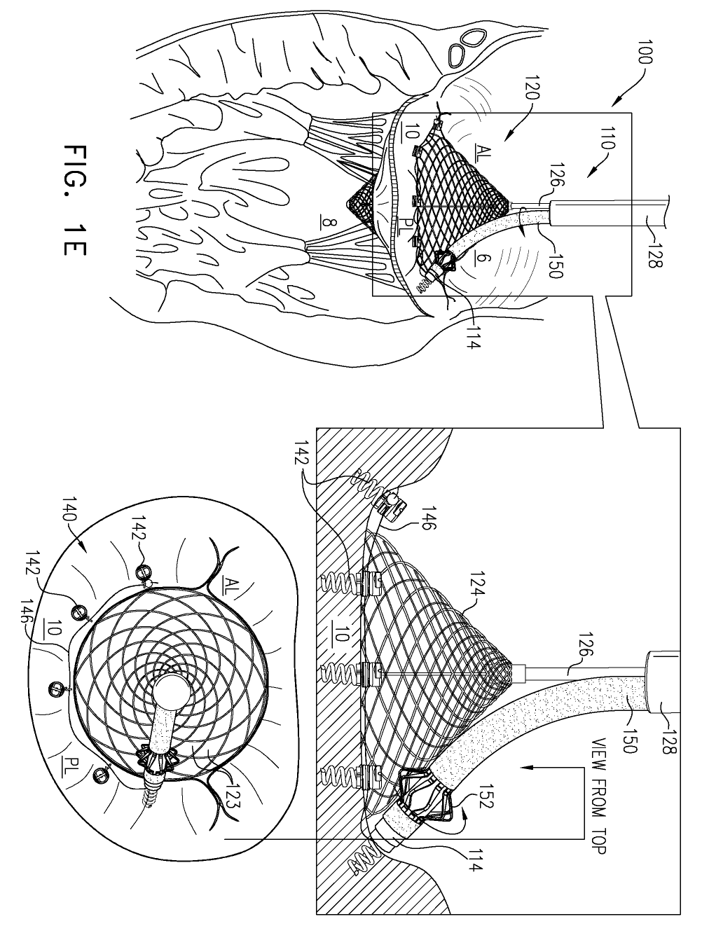

Reference is now made to FIGS. 1A-G, which illustrate a system 100 comprising a delivery assembly 110 that is adapted to facilitate the implantation of an implant 140 (e.g., an annuloplasty implant) circumferentially around an annulus 10 of a heart. For example, implant 140 can comprise a series of anchors 142 (e.g., sutures, helical anchors, staples, darts, clips, pledget anchors and/or toggle anchors), for anchoring to tissue of a valve annulus (e.g., into tissue of the annulus and/or into tissue of leaflets of the valve). In some implementations, the implant comprises a tether 146 that tethers the series of anchors 142 to one another, such that anchoring the series of anchors around the tissue positions the tether circumferentially along the valve annulus (e.g., along an atrial surface thereof). The implant may therefore be an annuloplasty implant. The tether can then be subsequently tensioned (e.g., contracted), in order to contract the tissue. For example, this contraction may be used to circumferentially reduce the size of the valve annulus.

Although FIGS. 1A-G show delivery assembly 110 being used to facilitate the positioning of an implant along tissue, it is to be to understood that a delivery assembly similar to that described herein could be used to facilitate the administration of other tissue treatments (e.g., implants and non-implants), for example, the administration of energy and/or a substance along tissue of a subject (e.g., a living subject and/or a simulation). Similarly, although the valve shown is illustrated as a mitral valve (having a posterior leaflet PL and an anterior leaflet AL), delivery assembly 110 may similarly be used at another valve of the heart, such as the tricuspid valve. Furthermore, it is to be understood that delivery assembly 110 could be used to implant at and/or treat heart tissues other than tissue of a heart valve, cardiovascular tissue other than tissue of the heart, and/or tissue that is outside the cardiovascular system (e.g., gastrointestinal tissue).

Delivery assembly 110 can comprise a guide assembly 120. At a distal part of guide assembly 120 (e.g., at a distal part of delivery assembly 110), the guide assembly comprises a guide frame 124 that is positionable within a native valve of the heart (e.g., with an upstream section of the guide frame within atrium 6 upstream of the valve, and a downstream section of the guide frame within ventricle 8 downstream of the valve). The distal part of delivery assembly 110 can be transluminally (e.g., transfemorally) advanceable to the heart of the subject, and guide frame 124 may be expandable within the heart. Guide frame 124 may be coupled to a control shaft 126 (e.g., to a distal end of the control shaft) that is configured to facilitate positioning and/or expansion of the guide frame. During implantation of implant 140, guide frame 124 may be disposed at a distal end of a sheath 128, e.g., with control shaft 126 extending through a lumen of the sheath.

Guide assembly 120 can be advanced transluminally (e.g., transfemorally), via sheath 128 of delivery assembly 110, while guide frame 124 is in a contracted state, and, once deployed out of a distal end of the sheath, guide frame 124 is expanded within the heart. This expansion may be achieved merely by unconstraining the guide frame (e.g., by the guide frame being self-expanding), or by expanding the guide frame by applying an expanding force to the guide frame, such as with a mechanical actuator or a balloon. In the example shown, guide frame 124 is expanded by tensioning an actuating wire 125, causing axial compression and radial expansion of the guide frame. Guide frame 124 may be resilient (e.g., may be elastically expanded), such as being inherently biased to expand or inherently biased to contract, or may be merely flexible (e.g., may be plastically expanded).

In some implementations, guide frame 124 defines a framework of struts 122, such that, when expanded within the heart, a lattice-like structure defined by the struts is formed. In this manner, guide frame 124 may define multiple windows 123 (e.g., gaps) in-between the struts.

In some implementations, guide frame 124 can comprise and/or be formed from a braided filament (e.g., wire), the braided filament thereby defining struts 122.

For some applications, guide frame 124 is formed by cutting a stock material (e.g., is cut from a tube).

In some implementations, guide frame 124 may be formed from a metal (e.g., nitinol, stainless steel, and/or cobalt chrome). In some implementations, guide frame 124 may be formed from a polymer.

While expanded and situated at the native valve, guide frame 124 may push the leaflets of the valve away from each other, and may result in the guide frame pressing against the tissue of the annulus. Nonetheless, the valve may continue to function at least in part, e.g., because guide frame 124 is open and allows blood flow therethrough, and/or because leaflets AL and PL remain partially functional (e.g., downstream of the guide frame), providing a net one-way flow of blood through the valve that may be sufficient for the duration of the procedure. In some implementations, guide frame 124 comprises one or more valve members therewithin, such as prosthetic leaflets, to provide temporary valve functionality during the procedure. In some implementations, leaflets AL and PL are disabled completely (e.g., due to placement of the guide frame), e.g., in implementations in which the guide frame is used at a tricuspid valve of the heart.

In some implementations, while guide frame 124 is in its expanded state, its downstream section may be tapered, such that the guide frame can be advanced, from atrium 6, in a downstream direction through the valve until it (e.g., a wider and/or flared section of the guide frame) comes to rest against the upstream surface of the annulus of the valve.

In some implementations, delivery assembly 110 comprises a tool 150 (e.g., a catheter) that is adapted to position and guide the implantation of implant 140 along the tissue. For example, in some implementations, tool 150 is an anchor catheter, through which an anchor driver, adapted to anchor the series of anchors to the tissue, is advanced. In some implementations, tool 150 is adapted to facilitate the anchoring of each anchor of the series sequentially along the valve annulus, such that the implant assumes an arc along the annulus.

In some implementations, during the implantation of implant 140 along the annulus, the system advantageously includes a rack-and-pinion mechanism between tool 150 and guide frame 124, which facilitates guidance of the tool around the annulus. In some implementations, tool 150 comprises one or more teeth 152 (e.g., radial teeth), adapted to engage the lattice (e.g., windows 123) of guide frame 124. In some embodiments, the tool 150 comprises multiple radial teeth 152, such that rotation of tool 150 around a central axis ax1 of the tool 150, drives (e.g., rolls) the tool 150 circumferentially along (e.g., around) the guide frame 124, e.g., with radial teeth 152 serving as a pinion and the lattice serving as a rack. This advantageously can facilitate controlled advancement of tool 150 around the annulus and/or stabilization of the tool 150 during anchoring of each of the anchors.

In the example shown, the rotation of tool 150 around guide frame 124 is achieved by the revolution of the tool 150 around central axis ax1 of the tool, e.g., such that radial teeth 152 revolve along with the tool 150, around the guide frame 124. For example, this may be achieved by rotating tool 150 from outside of the subject. However, it is to be understood that a mechanism in which radial teeth 152 revolve around central axis ax1 while part of tool 150 remains non-rotating can also be achieved.

FIGS. 1A-G represent a series of steps that may be performed by the operator, to circumferentially reduce the size of an annulus 10 of a heart valve, in accordance with some applications. Although FIGS. 1A-G show a sequence of at least some steps in a procedure, and may, in fact, be performed in the order shown, these figures are also intended to illustrate the capability of system 100, independently of any particular sequence of steps.

FIG. 1A shows guide frame 124 deployed within the heart (e.g., within a valve orifice of a valve of the heart), such that the guide frame abuts the annulus along a midsection (e.g., a circumference) of the guide frame. As shown in FIG. 1A, tool 150 can be transluminally (e.g., transfemorally) delivered to the heart (e.g., within sheath 128) with teeth 152 in a retracted state (e.g., lying flat against the tool, flush with a surface of the tool, or disposed within the tool), and once deployed out of the sheath (e.g., and against the guide frame, FIG. 1B), the teeth can be extended radially outward in order to engage the lattice (e.g., by protruding into windows 123) of guide frame 124 (FIG. 1C).

In some implementations, delivery assembly 110 is adapted to press tool 150 (e.g., teeth 152 thereof) against guide frame 124, in order to maintain engagement of teeth 152 with the guide frame. In some implementations, and as shown, this is achieved, at least in part, by both guide frame 124, and tool 150, extending transluminally through sheath 128 and out of a distal end of the sheath, e.g., such that the sheath biases the tool against the guide frame. In some implementations, this may be further facilitated by tool 150 being biased toward being straight.

Implant 140 can then be implanted at the heart (FIGS. 1C-E), facilitated by the rack-and-pinion mechanism between tool 150 and guide frame 124. For example, as tool 150 is driven circumferentially around the annulus, using the rack-and-pinion mechanism, a user (e.g., a physician) may pause the driving of the tool to anchor (e.g., secure) anchors 142 at various points along the circumference. System 100 may thus advantageously provide enhanced control of the positioning of a tool along a tissue (such as tissue of a valve annulus). In some embodiments, the enhanced control of the positioning of the tool may be due to the geometric relationship between the number of rotations of the tool and the distance it moves along the guide frame, and/or due to stabilization of the tool against the guide frame.

In some implementations, guide frame 124 may have predefined “stop” points defined by the guide frame. These “stop” points may be provided to the user via haptic feedback. For instance, at these “stop” points along the circumference, the guide frame defining struts may provide resistance to the user of the apparatus and/or system such that it is harder for teeth 152 to revolve around the guide frame 124.

At a point along the circumference in which it is determined that an anchor 142 is to be anchored (e.g., at a “stop” point along the guide frame), anchor driver 114 may drive an anchor 142 into the tissue. For some applications, anchor driver 114 is advanced, while engaged with the anchor, transluminally through tool 150, to the tissue (e.g., until anchor driver 114 and/or the anchor abuts the tissue), wherein the anchor can then be anchored to tissue of the annulus. As described hereinabove, each anchor 142 of the series can be anchored to the tissue while tether 146 extends (e.g., is threaded onto) the anchor, such that the tether becomes disposed circumferentially (e.g., in an arc), along the annulus.

Once it has been determined that a sufficient amount of anchors 142 of the series have been anchored to the tissue, as shown in FIG. 1F, tool 150 and guide frame 124 can then be withdrawn from the heart (e.g., via sheath 128), leaving implant 140 implanted therearound (e.g., such that implant 140 is in an implanted state). In some implementations, tether 146 is tensioned (e.g., prior to the withdrawal of sheath 128), in order to circumferentially contract the size of the annulus, thereby reducing valve regurgitation (FIG. 1G).

A stopper 148 (e.g., a lock) may be locked to tether 146 in order to lock in the applied tension.

Although FIGS. 1A-G illustrate an implementation in which tool 150 is driven along an exterior surface of guide frame 124 (e.g., thereby utilizing an “external gear” mechanism between the tool and the guide frame), it is to be noted that an “internal gear” mechanism between the tool and the guide frame could similarly be utilized, in which the tool is driven along an interior surface of the guide frame. For example, guide frame 124 could be expanded within a blood vessel of a subject, e.g., such that an exterior surface of the guide frame contacts the blood vessel wall, and tool 150 could then be driven along the interior surface of the guide frame (e.g., such that at least teeth 152 of the tool are disposed within the guide frame during driving of the tool along the guide frame).

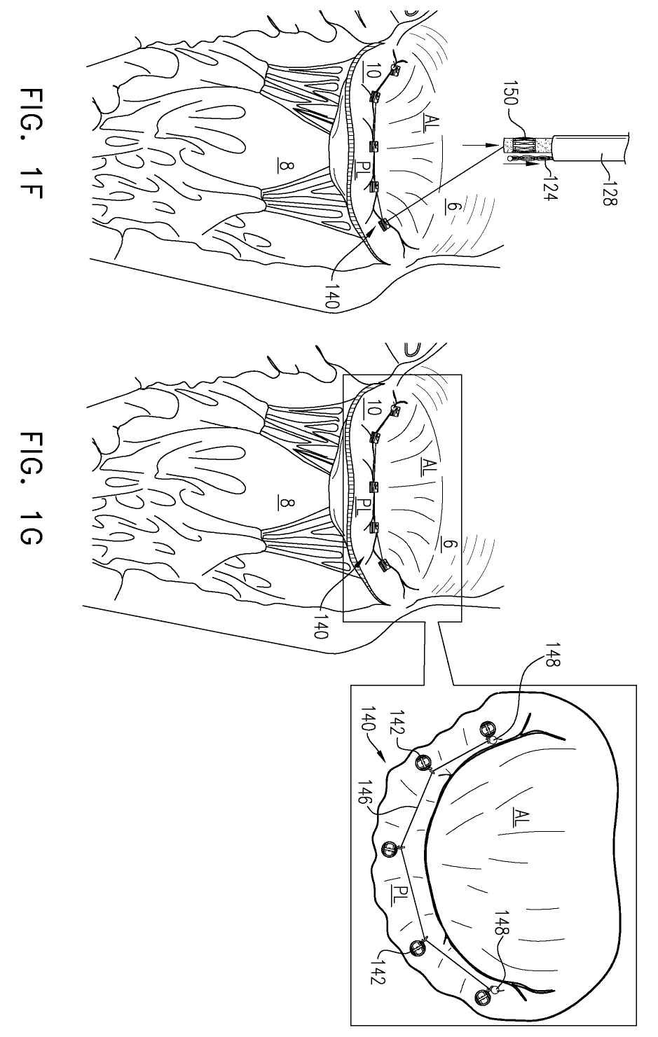

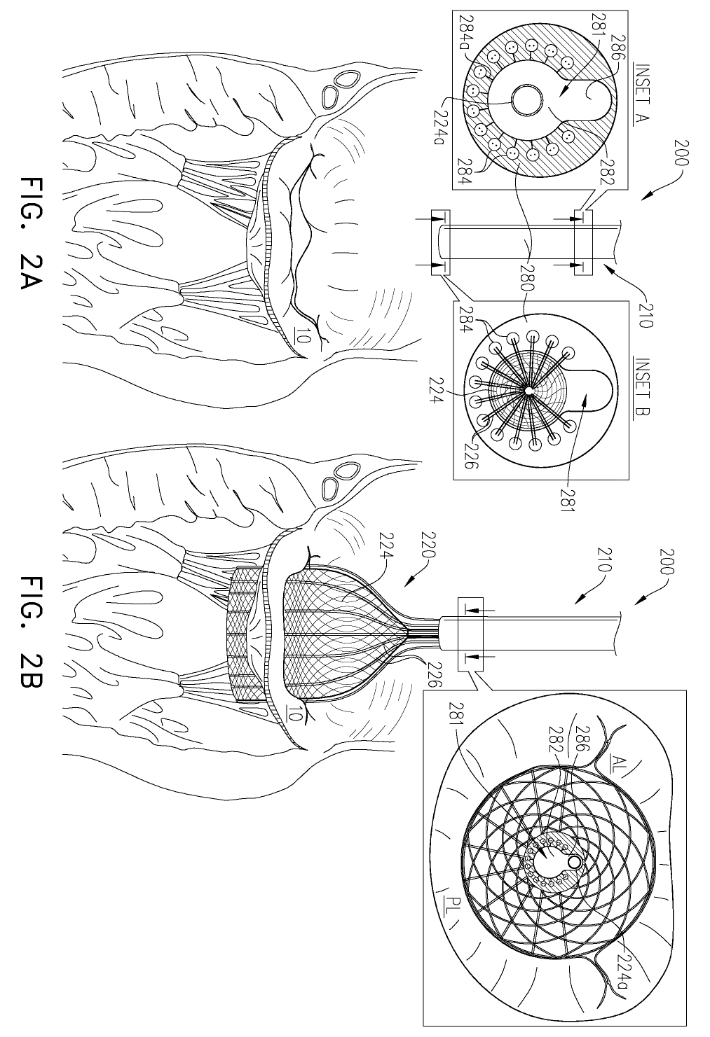

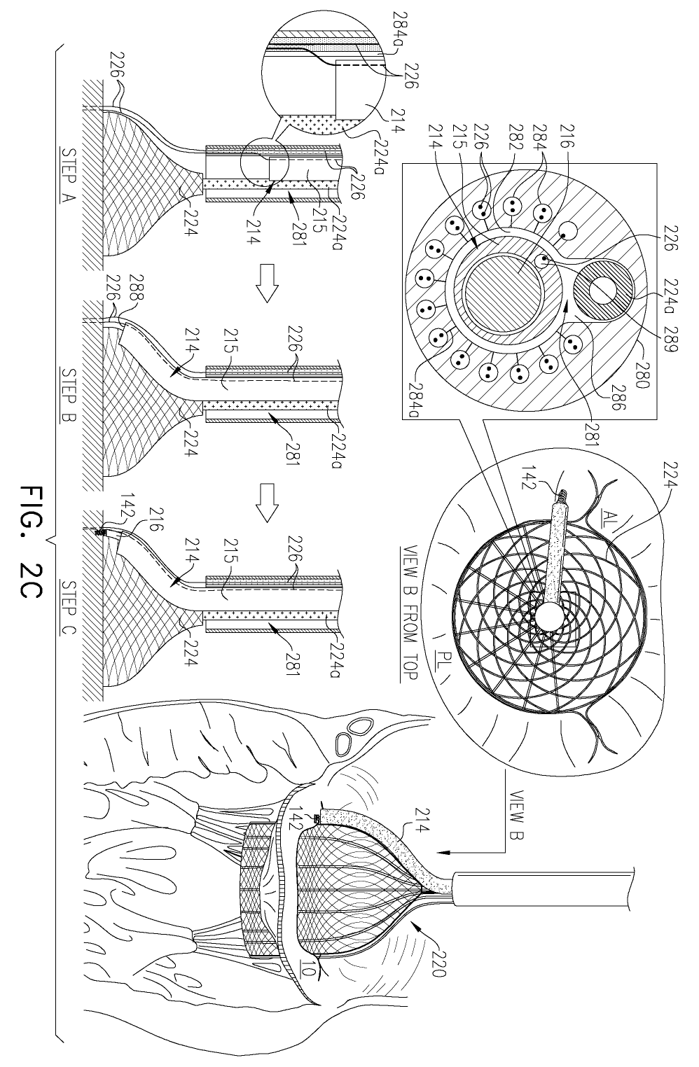

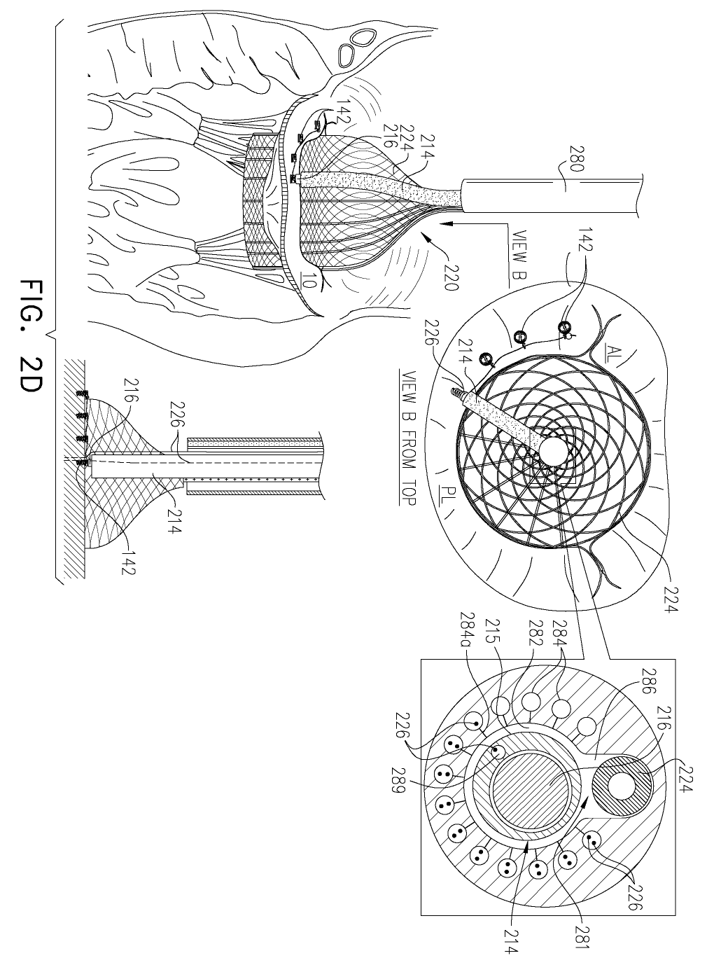

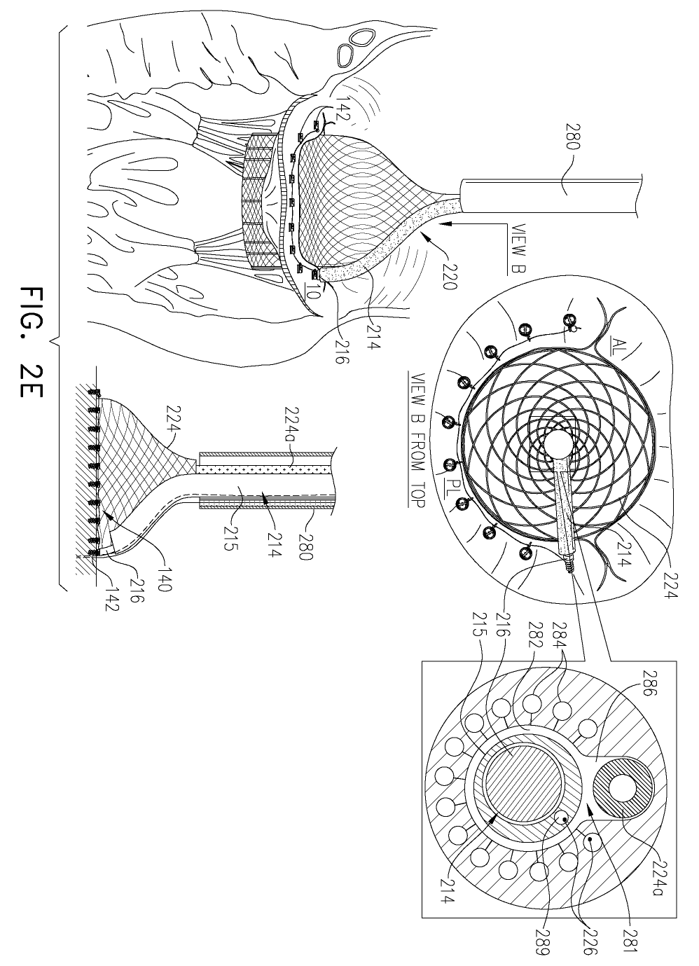

Reference is now made to FIGS. 2A-G, which show a system 200 comprising a delivery assembly 210 that comprises a guide assembly 220, for guiding an implant (e.g., implant 140, as shown, or a variant thereof) circumferentially around an annulus 10 of a valve of a subject (e.g., a living subject and/or a simulation). In some implementations, delivery assembly 210 may be a variant of delivery assembly 110, e.g., delivery assembly 210 comprises a guide assembly 220 that may be a variant of guide assembly 120. Similarly to guide assembly 120, at a distal part of guide assembly 220 (e.g., at a distal part of delivery assembly 210), the guide assembly comprises a guide frame 224 that is positionable within a native valve of the heart (e.g., within a valve orifice of the valve, such that an upstream section of the guide frame is disposed within atrium 6 upstream of the valve, and a downstream section of the guide frame is disposed within ventricle 8 downstream of the valve). In some implementations, guide frame 224 does not taper inwardly.

Although FIGS. 2A-G show delivery assembly 210 being used to facilitate the positioning of an implant along tissue, it is to be understood that a delivery assembly similar to that described herein could be used to facilitate the administration of other tissue treatments (e.g., implants and non-implants), for example, the administration of energy and/or a substance along tissue of a subject. Similarly, although the valve shown is illustrated as a mitral valve (having a posterior leaflet PL and an anterior leaflet AL), delivery assembly 210 may similarly be used at another valve of the heart, such as the tricuspid valve. Furthermore, it is to be understood that delivery assembly 210 could be used to implant at and/or treat heart tissues other than tissue of a heart valve, cardiovascular tissue other than tissue of the heart, and/or tissue that is outside the cardiovascular system (e.g., gastrointestinal tissue).

In some implementations, and as shown in FIG. 2B, a series of guides 226 (e.g., wires) extend, from outside of the subject, transluminally through delivery assembly 210 to the heart, where the guides extend longitudinally along an exterior of guide frame 224, e.g., to a respective coupling point wherein each guide is coupled to the guide frame at a part of the guide frame that is positioned downstream of the valve. That is, the guide frame can be positioned, within the heart, adjacent the tissue, with the guides extending longitudinally along the guide frame.

In some implementations, anchors 142 are anchored to the heart by transluminally (e.g., transfemorally) sliding the anchor through delivery assembly 210 (e.g., through a primary lumen 281 of a sheath 280 thereof) and along a corresponding guide 226, until the anchor reaches tissue of annulus 10 (FIG. 2C). As shown, and as described in more detail hereinbelow, this sliding may be achieved by sliding an anchor tool 214 of delivery assembly 210 over and along the corresponding guide, while the anchor tool is engaged with the anchor. In such implementations, the anchors may become automatically disconnected from their corresponding guides upon the anchor being disengaged from anchor tool 214.

In some implementations (not shown), the anchor is slid over and along the corresponding guide (e.g., the anchor is threaded onto the guide). Once the anchor has been anchored to the tissue, the anchor is detached from the corresponding guide, and the guide can then be withdrawn from the heart.

Once the anchor is disposed at the tissue of the annulus, the anchor can be anchored (e.g., secured) to the tissue, e.g., adjacent the guide frame. In this manner, annulus 10 advantageously acts as a shelf or shoulder that anchor 142 will meet (i.e., before reaching the end of guide 226), thereby advantageously desensitizing the system to the position (e.g., depth) of guide frame 224 within the valve. This may thus obviate the need to ensure that a particular part of the guide frame (e.g., a section around which the anchors will be implanted) is positioned uniformly along the native valve. This may be particularly advantageous for tissue surfaces that are not flat, e.g., for the saddle-shape of the annulus.

In some implementations, anchor tool 214 comprises an anchor driver 216 that is adapted to engage with the anchors of the implant, and to anchor the anchors to tissue of annulus 10. In some implementations, and as shown, anchor tool 214 further comprises an anchor catheter 215 through which the anchor driver extends. In some implementations, anchor catheter 215 is advanced over and along a corresponding guide 226 to the tissue. In some such implementations, this is achieved by the anchor catheter defining a guide lumen 289, that can be threaded over the corresponding guide. In some implementations, and as shown, guide lumen 289 is eccentric. Once anchor catheter 215 is positioned at the tissue, anchor driver 216 (e.g., disposed within a driver lumen of the anchor catheter) can then be used to anchor the anchor to the tissue. In some implementations, the driver lumen of the anchor catheter is also eccentric, e.g., as shown. In some implementations, the anchor catheter and the anchor driver are advanced together, e.g., anchor tool 214 is advanced as a unitary device. In some implementations, the anchor driver is advanced through the anchor catheter after the anchor catheter has been advanced over and along guide 226. In some implementations, the eccentricity of the guide lumen and/or the driver lumen contributes to the predetermined spacing between each anchor and guide frame 224.

FIGS. 2A-G represent a series of steps that may be performed by the operator, to circumferentially reduce the size of an annulus 10 of a heart valve, in accordance with some applications. Although FIGS. 2A-G show a sequence of at least some steps in a procedure, and may, in fact, be performed in the order shown, these figures are also intended to illustrate the capability of system 200, independently of any particular sequence of steps.

In some implementations, sheath (e.g., catheter) 280 is transluminally advanced through vasculature of a subject, to the heart of the subject (FIG. 2A). In some implementations, guide frame 224 is advanced to the heart within a major lumen region 282 of primary lumen 281 (e.g., in a compressed state), e.g., as is visible in inset B of FIG. 2A. Primary lumen 281 of sheath 280 may also have a minor lumen region 286, e.g., the primary lumen may be keyhole-shaped. In such implementations, during delivery of guide frame 224 to the heart, a control shaft 224a of the guide frame may extend, from guide frame 224, proximally through the primary lumen and out of the subject– e.g., as is visible in inset A of FIG. 2A. In some such implementations, guide frame 224 may be delivered to the heart while housed within a distal end portion of primary lumen 281, and/or while protruding distally out of the primary lumen.

As illustrated in the transition from FIG. 2A to 2B, once guide frame 224 is delivered out of sheath 280 (e.g., out of a distal end thereof) and into the heart, control shaft 224a, which may be narrower than the guide frame even when the guide frame is compressed, can be shifted laterally to minor lumen region 286 of the sheath. This may be achieved by pushing an extracorporeal portion of control shaft 224a laterally. This shifting may vacate major lumen region 282, providing space for anchor tool 214 to be advanced through the major region, e.g., as will be described with reference to FIGS. 2C-E. In some implementations, this shifting occurs, or is performed, independently of anchor tool 214. In some implementations, this shifting occurs as a result of advancing the anchor tool through major region 282, e.g., as the anchor tool is advanced distally into the major lumen region, the anchor tool progressively pushes shaft 224a aside, into minor region 286.

In some implementations, the above approach can also be used for sheath 128 of delivery assembly 110, mutatis mutandis. For example, sheath 128 may similarly define a primary lumen that has a major lumen region and a minor lumen region. In such implementations, guide frame 124 may be delivered via the major lumen region (e.g., while positioned at a distal end of the major lumen region), with control shaft 126 extending proximally through the major lumen region (e.g., and outside of the subject). In such implementations, once positioned at the heart, control shaft 126 may be shifted laterally into a minor lumen region of the primary lumen. Tool 150 may then be advanced through the vacated major lumen region, e.g., and subsequently be used to anchor anchors 142 along the tissue. As described hereinabove with reference to sheath 280, the shifting of control shaft 126 into the minor lumen region may occur as a result of advancing the tool 150 through the major lumen region– e.g., as the tool is advanced distally into the major lumen region, the tool progressively pushes shaft 126 aside, into the minor region.

Each guide 226 may loop around guide frame 224 (e.g., around a strut of the downstream section of the guide frame), and then extend proximally back along the guide frame (e.g., in a similarly longitudinal fashion), such that once the guide frame is deployed in the heart, each guide 226 has two parallel lengths that extend distally through sheath 280, out of the sheath, and along the guide frame, to a bight that loops around guide frame 224.

In some implementations, rather than having two parallel lengths of each guide extending longitudinally along the guide frame, a single length of each guide 226 may extend, from a coupling point at a downstream section of the guide frame, proximally alongside the guide frame (e.g., longitudinally along the guide frame), and optionally into delivery assembly 210 (e.g., and through sheath 280 to an extracorporeal end of the guide). In some such implementations, after implanting implant 140 along the tissue (e.g., facilitated by the guides), the guides may be withdrawn together with the guide frame from the heart, e.g., by withdrawing the guide frame back into sheath 280 while the guides remain coupled to the guide frame at their respective coupling points.

In some implementations, sheath 280 may define multiple secondary lumens 284. Each secondary lumen 284 houses a corresponding guide 226 during delivery of guide frame 224 to the heart. For example, and as shown in FIG. 2A, during delivery of sheath 280 to the heart, each guide 226 can extend, from a first end of the guide that is outside of the subject, through a corresponding secondary lumen 284, looping around a downstream section of the guide frame, and then back through the same secondary lumen to a second end of the guide that is disposed out of the subject. Alternatively, during delivery of sheath 280 to the heart, a single length of each guide may be housed in each secondary lumen.

Inset B of FIG. 2A shows the downstream section of the guide frame around which the bights of guides 226 are looped, and the two lengths of each guide extending from the downstream section of the guide frame into their corresponding secondary lumens 284. Inset A of FIG. 2A is a cross section through a more proximal part of sheath 280, showing the two lengths of each guide (as a pair of dots) within their corresponding secondary lumen 284. The use of secondary lumens 284 may advantageously prevent guides 226 from becoming tangled or ensnared with each other and/or with the anchors that will be subsequently advanced through the sheath, e.g., by keeping the guides out of primary lumen 281.

FIGS. 2C-E show anchor tool 214 anchoring anchors 142 circumferentially around the tissue. As described hereinabove, the advancement of anchor tool 214 may be performed by sliding the anchor tool over and along a guide 226, towards the heart. For example, in some implementations, anchor tool 214 may include a rapid exchange feature, such as a lateral and/or an oblique lumen at its distal end. For such implementations, a proximal end of a guide 226 can be threaded through the rapid exchange feature (rather than through the entire length of tool 214), and in that manner, the anchor tool can be advanced over and along the guide, through primary lumen 281 (e.g., through major lumen 282 thereof), towards the heart. In some implementations, and as described hereinabove, anchor catheter 215 may include a guide lumen 289 through which the corresponding guide can be threaded.

Each of secondary lumens 284 of the catheter may be connected, along its length, to primary lumen 281 via an elongate slit 284a, such that, as anchor tool 214 is advanced progressively distally through primary lumen 281, the anchor tool pulls guide 226 laterally through the corresponding slit and into the primary lumen (e.g., as illustrated by step A of FIG. 2C). Within the heart, further sliding of anchor tool 214 over and along the guide brings the anchor tool to tissue of the annulus (e.g., until anchor tool 214 and/or the anchor abuts a site on the annulus (step B, FIG. 2C)). As described above, in this manner, annulus 10 may advantageously act as a shelf for the anchor, e.g., by obstructing the anchor from being advanced further distally towards the coupling point of the guide to the guide frame. At this site on the annulus, anchor 142 can be anchored to the tissue by anchor tool 214 (e.g., anchor driver 216 thereof) applying an anchoring force to the anchor (step C, FIG. 2C).

Once anchor 142 has been anchored to the annulus, anchor tool 214 can be disengaged from the anchor and retracted from the heart, leaving the anchor anchored to the tissue. The guide 226 corresponding to that anchor may then be retracted, e.g., by releasing a first end of the guide, and pulling on the other end such that the first end is drawn distally through sheath 280 to guide frame 224, from which it unloops. In some implementations, the guide 226 is retracted concurrently with the retraction of anchor tool 214, e.g., such that the guide is withdrawn, along with the anchor tool, out of primary lumen 281 and the subject.

This process can be repeated for the other anchors of the series, e.g., (FIGS. 2D-E). In this manner, guides 226 may advantageously define the position and/or spacing along annulus 10 at which anchors 142 are anchored.

Once it has been determined that enough anchors 142 of the series have been anchored to the tissue, guide assembly 220 (e.g., guide frame 224) can then be withdrawn from the heart (e.g., via sheath 280), leaving implant 140 implanted around the annulus (FIG. 2F).

In some implementations, and as shown, a tether (e.g., tether 146) extends between the anchors, such that once the anchors have been implanted along the annulus, the tether is disposed along the tissue (e.g., along the annulus). In some such implementations, each anchor of the series may be delivered to the heart while the tether is coupled to the anchor (e.g., by extending through an eyelet of the anchor), such that anchoring the anchors along the tissue disposes the tether in an arc along the annulus. The tether can then be tensioned to reshape (e.g., to contract) the annulus (FIG. 2F). In some implementations, and as shown, tether 146 is tensioned prior while sheath 280 is still in place.

A stopper 148 (e.g., a lock) may then be locked to tether 146 in order to lock in the applied tension (FIG. 2G).

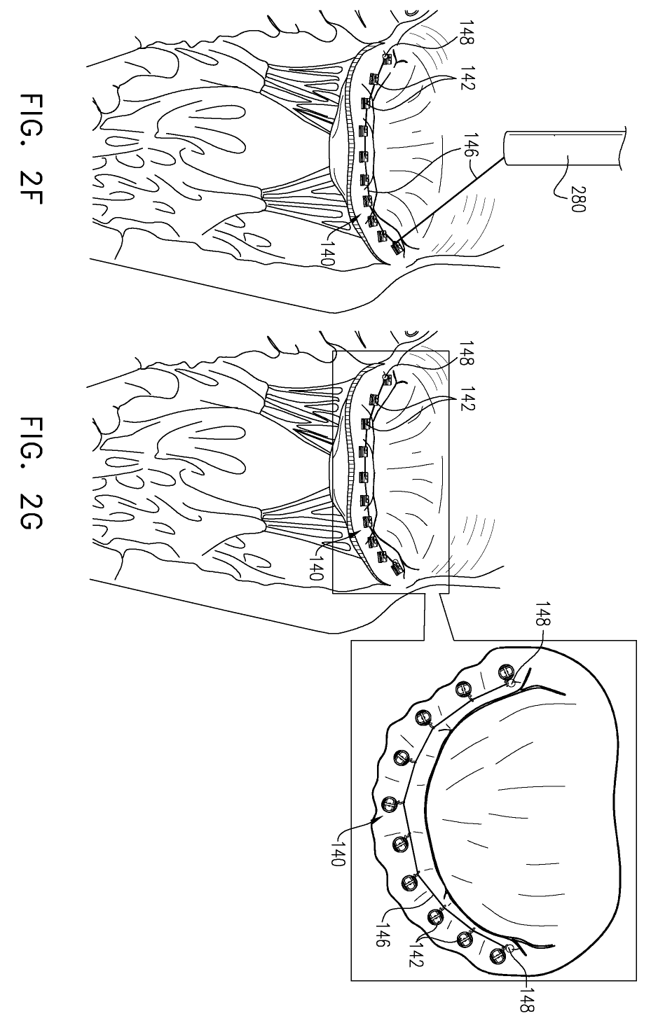

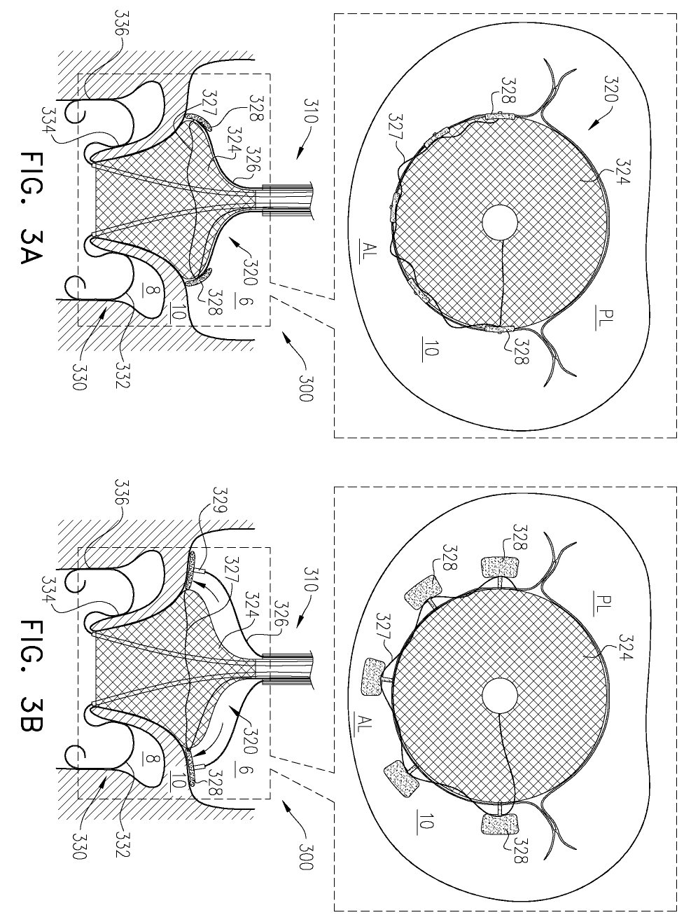

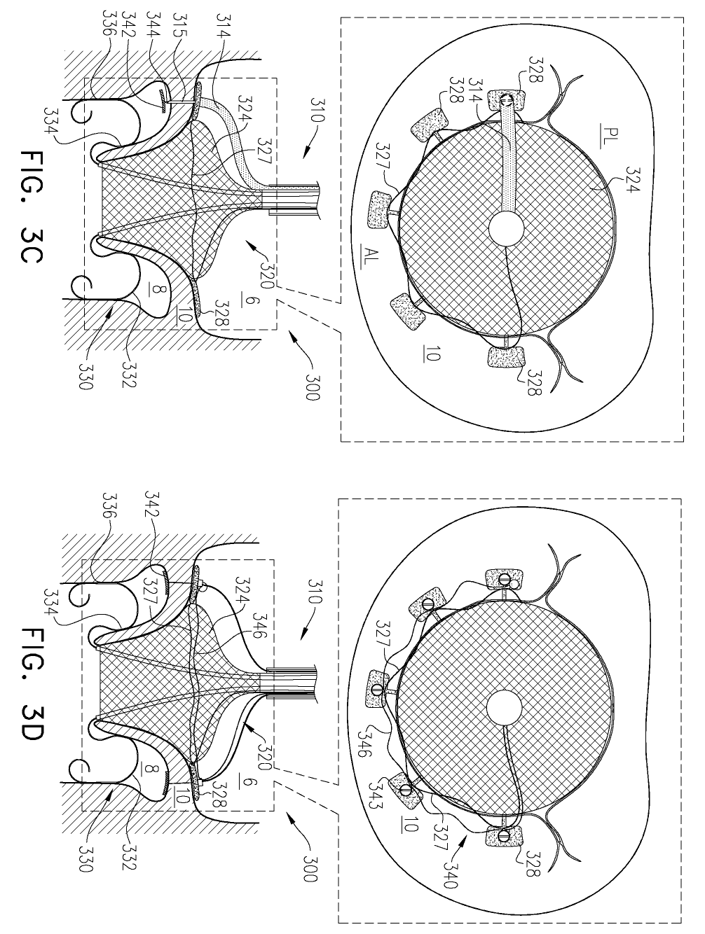

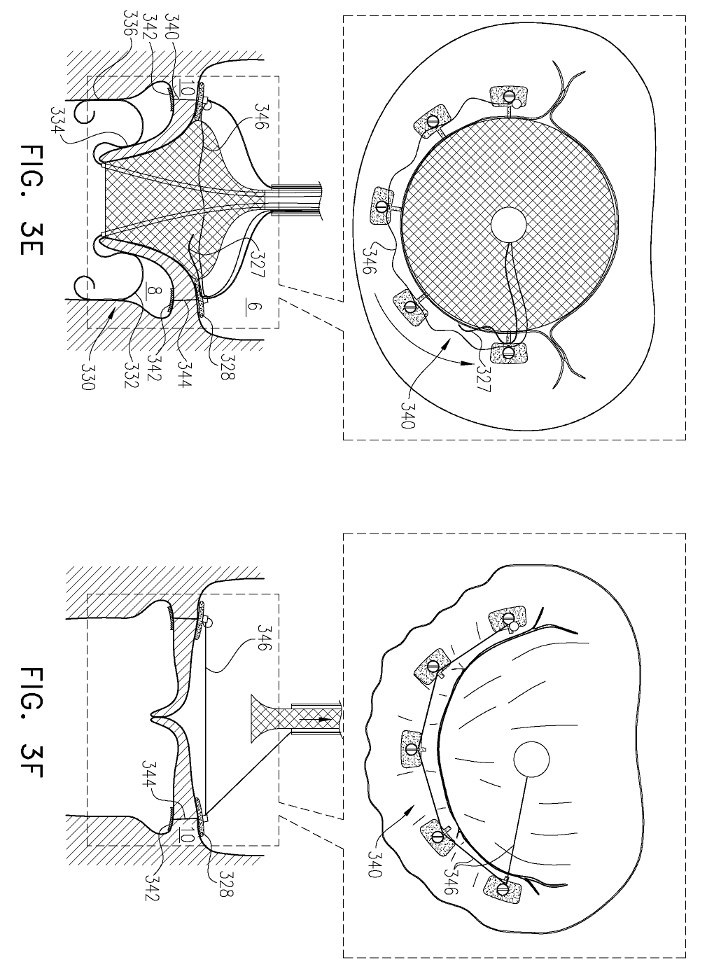

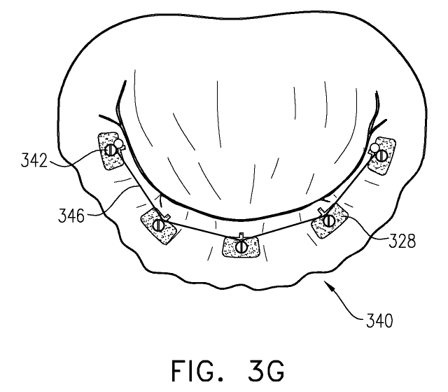

Reference is now made to FIGS. 3A-G, which show a system 300 comprising a delivery assembly 310 that comprises a guide assembly 320, for guiding an implant 340 circumferentially around an annulus 10 of a valve of a subject (e.g., a living subject and/or a simulation).

In some implementations, and similarly to implant 140, implant 340 comprises a tether 346 that tethers a series of anchors 342 to one another, such that anchoring the series of anchors around the valve positions the tether circumferentially along the valve annulus. The tether can then be subsequently tensioned (e.g., contracted), in order to contract the tissue. For example, this contraction may be used to circumferentially reduce the size of the valve annulus.

In some implementations, anchors 342 can be toggle anchors, helical anchors, pledget anchors and/or sutures.

In some implementations, delivery assembly 310 may be a variant of delivery assembly 110 and/or 210, e.g., delivery assembly 310 comprises a guide assembly 320 that may be a variant of guide assembly 120 and/or 220. Similarly to the guide assemblies described hereinabove, at a distal part of guide assembly 320 (e.g., at a distal part of delivery assembly 310), the guide assembly comprises a guide frame 324 that is positionable within a native valve of the heart (e.g., with an upstream section of the guide frame within atrium 6 upstream of the valve, and a downstream section of the guide frame within ventricle 8 downstream of the valve).

In some implementations, guide frame 324 has a series of flaps 328 attached around the midsection of the guide frame. A guide 326 (e.g., a variant of guides 226 described hereinabove) extends from each flap, proximally through delivery assembly 310, and out of the subject. Each flap 328 may be coupled to its corresponding guide 326 at a coupling point 329. The guide frame can be delivered to the heart with each flap 328 in a retracted position against guide frame 324. FIG. 3A shows guide frame after its delivery to the heart, with flaps 328 still remaining in their retracted positions. In some implementations, the flaps are maintained in their retracted position by maintaining tension on each guide 326, such as by pulling the guides proximally. Upon positioning of the guide frame at the native valve (e.g., such that a midsection of the guide frame abuts tissue 10 of the annulus), each flap 328 can be deflected away from the guide frame, e.g., such that the respective guide 326 becomes spaced (e.g., spaced further) from the guide frame away from the valve orifice (FIG. 3B). This deflection may place each flap 328 against the surface of the annulus, e.g., as shown. This deflection may be performed by actuating guides 326, e.g., by pushing distally on the guides and/or by releasing tension on the guides. In some implementations, flaps 328 may be constrained in their retracted positioned within sheath 280, and upon release from the sheath, may automatically deflect away from the guide frame. For example, the flaps may include a shape-memory material (e.g., a nitinol wire) that biases the flap to deflect away from the guide frame. In some implementations, and as shown, this deflection of flaps 328 causes guide assembly 320 to assume a flower-like appearance on the annulus, e.g., such that each flap resembles a petal extending radially outwards from guide frame 324, on the surface of the annulus, e.g., as shown in the inset of FIG. 3B. Each flap 328 may comprise one or more sheets, such as sheets of fabric or film. In some implementations, each flap may be padded.

In some implementations, a connecting wire 327 attaches each of flaps 328 to guide frame 324. For example, and as shown in FIG. 3C, connecting wire 327 can weave in and out of the guide frame, and, at each respective flap, be threaded through the flap (e.g., through an eyelet thereof). As described hereinbelow, in some implementations, subsequently to anchoring anchors 342 to the tissue, flaps 328 may become part of implant 340. In such implementations, prior to withdrawing the guide frame from the heart, e.g., post-implantation of implant 340, connecting wire 327 can be withdrawn in order to disengage the flaps from the guide frame, and/or guides 326 can be disconnected from flaps 328 to disengage the guides from the flaps.

In some implementations, once guide frame 324 is positioned within the valve orifice, with each flap 328 positioned over annulus 10, each anchor 342 of the series can then be delivered (e.g., slid) to the heart over and along a corresponding guide 326 (e.g., similarly to the “zipline” technique described hereinabove with reference to FIGS. 2A-G), until the anchor reaches its corresponding flap 328 (e.g., until the anchor abuts the flap that is at the distal end of that guide). The anchor can then be anchored to the tissue (FIGS. 3C-D). This technique may advantageously facilitate accurate positioning of each anchor – at a reliable and predetermined distance from the valve orifice, guided by the distance by which the respective flap positions the guide from the guide frame.

In some implementations, anchors 342 are toggle anchors. For example, the anchors may be delivered through the annulus (e.g., through a corresponding flap and through the annulus) into the ventricle 8 downstream of the annulus, such that, when deployed, the anchors are exposed within the ventricle and lie against a ventricular surface of the annulus (FIG. 3D). In implementations in which the anchors are toggle anchors, a cord 344 may extend, from anchor 342 within the ventricle, through annulus 10 and flap 328, to anchor head 343. In some implementations, flaps 328 serve as pledgets for the anchors.

In some such implementations, an anchor driver 314 may be used to deliver the anchors through the annulus and into ventricle 8. For example, each anchor 342 may be delivered to the heart while housed within a hollow needle 315 that is disposed at the distal end of anchor driver 314. Once needle 315 is positioned at the annulus (e.g., positioned against an atrial surface of the annulus), the needle may be pushed through the annulus, e.g., such that the needle traverses the annulus, and at least a distal tip of the needle becomes disposed in the ventricle. In some implementations, during delivery of anchor 342 to the annulus, a pusher (not shown) extends distally through anchor driver 314 and abuts a proximal end of the anchor, such that, once the distal end of the needle is disposed within the ventricle, the pusher can be used to push the anchor out of the needle and into the ventricle.

In some implementations, in order to keep the leaflets 16 of the valve clear, during anchoring, of such annulus-traversing anchors 342, a wire arrangement 330 may be temporarily positioned within the ventricle, to bias the leaflets of the valve medially (e.g., inwardly) away from ventricle wall and/or the subannular space/groove. For example, this may prevent anchors 342 from becoming anchored to the leaflets, e.g., by keeping the leaflets away from the ventricular side of annulus 10 through which the anchors pass. In some such implementations, wire arrangement 330 may comprise multiple legs 332 that extend, from guide assembly 320 (e.g., from a distal part of guide frame 324), and distally into the ventricle, such that, upon positioning of the guide assembly at (e.g., through) the valve, the legs protrude into the ventricle downstream of the valve being treated. In some implementation, one or more of legs 332 define a resilient portion 334 that can be more resilient (e.g., more flexible and resilient) than the rest of the leg, the wire arrangement being positionable in the ventricle such that the resilient portion presses against leaflet 16 (e.g., as shown in FIG. 3A). The resilience of resilient portion 334 can contribute to the function of wire arrangement 330, by pushing the leaflets medially and away from the ventricular wall. In some applications, and as shown, when wire arrangement 330 is so positioned, a contact-portion 336 of leg 332 contacts (e.g., abuts or otherwise atraumatically contacts) tissue of ventricle 8. For some such applications, and as shown, leg 332 is shaped (e.g., curved) such that its distal tip does not contact tissue of the heart.

FIGS. 3A-G represent a series of steps that may be performed by the operator, to circumferentially reduce the size of an annulus 10 of a heart valve, in accordance with some applications. Although FIGS. 3A-G show a sequence of at least some steps in a procedure, and may, in fact, be performed in the order shown, these figures are also intended to illustrate the capability of system 200, independently of any particular sequence of steps.

FIG. 3A shows guide assembly 320 positioned within the valve to be treated. In some implementations, guide assembly 320 can be delivered via a sheath (e.g., catheter) transluminally through vasculature of a subject, to the heart of the subject, while the guide frame is constrained within a compressed state within the sheath. Once guide frame 324 has been deployed from out of the sheath, the guide frame is expanded (e.g., by exposing the guide frame out of the sheath, and the guide frame responsively self-expanding, or via mechanical expansion of the guide frame). As shown, at this stage, each flap 328 may lie against guide frame 324, e.g., due to tension on each corresponding guide 326.

In implementations in which wire arrangement 330 is used, legs 332 may already be positioned within the ventricle at this stage, e.g., by extending the legs distally from out of guide assembly 320 (e.g., from out of guide frame 324). As described hereinabove, wire arrangement 330 can be positioned to bias leaflets 16 medially inwards in preparation for annulus-traversing anchors 342.

Flaps 328 can then be defected away from the guide frame, such that each flap extends outwardly from the guide frame, away from the valve orifice, and/or against the surface of the annulus (FIG. 3B). This may be achieved by actuating guides 326 (e.g., by pushing distally on the guides and/or by releasing tension on the guides).

An anchor 342 (e.g., a toggle anchor), can then be delivered towards the heart, by sliding the anchor (e.g., by sliding an anchor driver 314 that is engaged with the anchor) over and along its corresponding guide, through the vasculature of the subject, into the heart and alongside guide frame 324, until the anchor and/or driver 314 reaches its corresponding flap (e.g., at the atrial surface of the annulus). In some implementations, for each anchor of the series, the spacing of respective coupling point 329 on each flap 328 may determine the distance that that anchor will be anchored from the valve orifice.

In some such implementations, anchor driver 314 may include an eyelet and/or a rapid exchange feature (e.g., an oblique lumen at its distal end), to allow for sliding of the anchor driver over guides 326.

Once at the annulus (e.g., once positioned against the atrial surface of the annulus), anchor driver 314 can be used to anchor the anchor to the tissue. In some implementations, each anchor 342 is anchored to the annulus in a manner that sandwiches its corresponding flap 328 between the anchor (e.g., an anchor head 343 thereof), and the tissue.

As described hereinabove, in some implementations, the anchor may be pushed through the annulus and into the ventricle while housed within needle 315 (FIG. 3C), such that the anchor becomes disposed within the ventricle (e.g., such that the anchor lies against a ventricular surface of the annulus). In such embodiments, cord 344 can extend, from anchor 342 within ventricle 8, through annulus 10 and flap 328, to anchor head 343.

This anchoring process can be repeated for some or all of flaps 328, until it has been determined that sufficient anchors 342 have been implanted around the valve (FIG. 3D).

In some implementations, flaps 328 are intracardially disengageable from the guide frame, to allow for withdrawal of the guide frame from the heart, leaving the flaps remaining as part of implant 340. In some implementations, this disengagement is achieved by retracting connecting wire 327, e.g., once the anchors have been anchored to the tissue. This can be achieved by pulling the connecting wire proximally, such that the flaps become disengaged from guide frame 324 (FIG. 3E).

In some such implementations, once implantation of implant 340 is complete, for each anchor of the series, flap 328 lies against an atrial surface of the annulus (e.g., defining an upstream component of the anchor, and/or serving as a pledget for the anchor), and ventricular anchor 342 lies against a ventricular surface of the annulus (thereby defining a downstream component of the anchor), with cord 344 connecting these two components to each other, each anchor thereby sandwiching tissue of annulus 10 therebetween.

In some implementations, anchors 342 are not anchored to flaps 328 and, once the anchors are anchored to the tissue, the flaps are subsequently removed from the heart. In such embodiments, flaps are used to predefine the distance each anchor is anchored from the valve orifice, but once used for that function, can be disengaged from the anchor (e.g., from around the anchor), and removed from the heart, leaving the anchors anchored uniformly anchored along the annulus.

In some implementations, and as described hereinabove, tether 346 extends along anchors 342, such that once the anchors have been implanted along annulus 10, the tether can be tensioned to reshape (e.g., contract) the annulus (FIG. 3F).

In some implementations, the contraction of the tissue is facilitated by contracting guide frame 324 medially while the guide frame remains attached to anchors 342 (e.g., via connecting wire 327), which themselves are anchored to the tissue. This contraction may be performed separately (e.g., before) or concurrently with tensioning of tether 346. The guide frame can then be detached from the implant (e.g., by withdrawing the connecting wire), optionally further contracted, and withdrawn from the heart.

The tension on tether 346 may be fixed by applying a lock or a crimp to the tether. Excess tether may then be cut and removed.

Once the implant has been tensioned, delivery assembly 310 can be withdrawn from the heart, leaving implant 340 implanted along the tissue (FIG. 3G).

Reference is again made to FIGS. 1A-G, 2A-G, and 3A-G. Although the figures of this disclosure illustrate an implant being implanted along a mitral valve of a heart, it should be understood that the techniques and systems described hereinabove can also be used for valves other than the mitral valve, for example, for the tricuspid valve of the heart. Furthermore, the techniques may be used for parts of the heart other than a valve, e.g., for ventriculoplasty.

Reference is again made to FIGS. 1A-G, 2A-G, and 3A-G. For each of the systems disclosed herein, its components are interchangeable, mutatis mutandis, with those of other systems disclosed herein. For example, the anchor driver and/or anchors of one system may be substituted with those of another system, mutatis mutandis. For example, implant 140 could be used with delivery assembly 310, and/or implant 340 could be used with either of delivery assemblies 110 or 210.

Furthermore, the systems, apparatus, and methods (and/or parts thereof) described herein may be modified to include and/or utilize any of the systems, apparatus, and methods (and/or parts thereof) disclosed in any of the following publications, which are incorporated herein by reference:

US Patent Application Publication 2012/0022557 to Cabiri et al.

US Patent Application Publication 2014/0309661 to Sheps et al.

US Patent Application Publication 2015/0272734 to Sheps et al.

US Patent Application Publication 2018/0049875 to Iflah et al.

US Patent Application Publication 2021/0145584 to Kasher et al.

US Patent Application Publication 2013/0190863 to Call et al.

US Patent Application Publication 2019/0254677 to Cahalane et al.

US Patent Application Publication 2019/0247037 to Groothuis et al.

International Patent Application Publication WO 2022/064401 to Halabi et al.

International Patent Application Publication WO 2022/172149 to Shafigh et al.

For example, implants 140 and/or 340 (or components thereof), and/or anchor drivers 114 and/or 216 may be modified to include one or more features or components of the implants and/or anchor drivers described in one or more of these references, mutatis mutandis. Similarly, the scope of the present disclosure includes modifying any of the systems or apparatus (e.g., the implants, delivery tools, and/or anchor drivers) described in these references to include one or more features or components of the systems or apparatus (e.g., the implants, delivery tools, and/or anchor drivers) described hereinabove.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.). The scope of the present disclosure includes, in some implementations, sterilizing one or more of any of the various systems, devices, apparatuses, etc. in this disclosure.

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., demonstrating in the air on an imaginary heart), etc.

Various implementations of systems, devices, methods, etc. are disclosed herein, and any combination of their features, components, and options can be made unless specifically excluded. For example, various descriptions of an implant can be used with any appropriate delivery assembly, and/or delivered and implanted by any appropriate method, even if a specific combination is not explicitly described. Likewise, the different constructions and features of devices and systems can be mixed and matched, such as by combining any guide rail type/feature, guide frame type/feature, anatomical site, etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

The present disclosure is not limited to the examples that have been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description.

Example Applications (some non-limiting examples of the concepts herein are recited below):

Example 1. A system for use with tissue of a cardiovascular system of a real or simulated subject, the system comprising: a delivery assembly comprising: (i) a guide frame, the delivery assembly configured to position the guide frame adjacent the tissue, and/or (ii) a tool comprising radial teeth adapted to engage the guide frame such that rotation of the tool drives the tool along the guide frame, the tool being configured to guide a treatment of the tissue via the driving of the tool along the guide frame.

Example 2. The system according to example 1, wherein: (i) the guide frame has an exterior surface and an interior surface, (ii) the delivery assembly is configured to position the guide frame such that the exterior surface faces the tissue, and/or (iii) the tool is configured such that the rotation of the tool drives the tool along the interior surface of the guide frame.

Example 3. The system according to any one of examples 1-2, wherein: (i) the guide frame has an exterior surface and an interior surface, (ii) the delivery assembly is configured to position the guide frame such that the exterior surface faces the tissue, and/or (iii) the tool is configured such that the rotation of the tool drives the tool along the exterior surface of the guide frame.

Example 4. The system according to any one of examples 1-3, wherein: (A) the delivery assembly comprises a sheath that defines: (i) a major lumen region, and/or (ii) a minor lumen region that has a smaller cross-sectional area than the major lumen region; (B) the delivery assembly is configured to transluminally advance the guide frame into the cardiovascular system while: (i) the guide frame is disposed within the major lumen region of the sheath, and/or (ii) a control shaft coupled to the guide frame extends, from the guide frame, proximally through the major lumen region; and/or, (C) while the guide frame remains distally outside of the sheath and the control shaft remains coupled to the guide frame, the control shaft is shiftable laterally out of the major lumen region and into the minor lumen region, such that the tool is advanceable through the major lumen region to the guide frame.

Example 5. The system according to any one of examples 1-4, wherein the guide frame is intracorporeally expandable at the tissue.

Example 6. The system according to any one of examples 1-5, wherein the tool comprises a catheter, configured to guide the treatment of the tissue by guiding a treatment assembly that is advanceable through the catheter.

Example 7. The system according to any one of examples 1-6, wherein the delivery assembly is configured to bias the tool against the guide frame.

Example 8. The system according to example 7, further comprising a sheath, wherein the system is configured to extend both the tool and the guide frame distally out of the sheath at the tissue in a manner that biases the tool against the guide frame.

Example 9. The system according to any one of examples 1-8, wherein the radial teeth are intracorporeally transitionable into a protruding state in which the teeth protrude radially outward from a surface of the tool.

Example 10. The system according to example 9, wherein the tool is advanceable toward the tissue while the radial teeth are in a retracted state in which the radial teeth lie flush with the surface of the tool.

Example 11. The system according to any one of examples 1-10, wherein the guide frame comprises struts that define windows therebetween, and/or wherein the radial teeth are adapted to engage the guide frame by the teeth protruding into the windows of the guide frame.

Example 12. The system according to example 11, wherein the struts are arranged in a lattice-like framework.

Example 13. The system according to any one of examples 1-12, wherein the system further comprises an implant, and/or wherein the tool is configured to guide the treatment of the tissue by guiding the implantation of the implant at the tissue.

Example 14. The system according to example 13, wherein the implant is an elongate implant, and/or wherein the tool is configured to guide the implantation of the implant alongside the guide frame.

Example 15. The system according to example 13, wherein the implant is an elongate implant, and/or wherein the tool is configured to guide the implantation of the implant along the tissue.

Example 16. The system according to example 15, wherein: (i) the tissue is tissue of an annulus of a heart of the subject, the annulus circumscribing an orifice of a heart valve, (ii) the elongate implant is an annuloplasty implant comprising a series of anchors, (iii) the delivery assembly is configured to position the guide frame adjacent the tissue by positioning the guide frame through the orifice such that the guide frame is adjacent the tissue of the annulus, and/or (iv) the tool is configured to guide the treatment of the tissue by guiding the implantation of anchors of the series of anchors around the annulus, such that the implant extends along the annulus.

Example 17. The system according to example 16, wherein the anchors of the series of anchors are connected via a tether, and/or wherein the tool is configured to guide the treatment of the tissue by guiding the implantation of the anchors around the annulus, such that the tether extends along the annulus.

Example 18. The system according to example 17, wherein the delivery assembly is configured to tension the tether to reduce a circumference of the annulus.

Example 19. The system according to example 16, wherein the tool comprises a catheter, transluminally advanceable to the heart, and/or wherein the system further comprises an anchor driver, adapted to, for an anchor of the series of anchors, engage the anchor and to deliver the anchor to the heart, via the catheter.

Example 20. A method for use at a tissue of a real or simulated subject, the method comprising: (A) advancing, into the real or simulated subject: (i) a guide frame, and/or (ii) a tool configured to deliver a treatment to the tissue, the tool comprising radial teeth, (B) positioning the guide frame adjacent the tissue, and/or (C) driving the tool along the guide frame by rotating the tool while the radial teeth are engaged with the guide frame.

Example 21. The method according to example 20, the method further comprising delivering a treatment along the tissue, facilitated by the driving of the tool along the guide frame.

Example 22. The method according to example 21, wherein delivering the treatment along the tissue comprises delivering the treatment out of the tool and along the tissue.

Example 23. The method according to example 21, wherein the tissue is tissue of an annulus of a valve of a heart of the real or simulated subject, and/or wherein delivering the treatment along the tissue comprises contracting a circumference of the annulus.

Example 24. The method according to any one of examples 20-23, wherein the method further comprises intracorporeally expanding the guide frame, subsequent to advancing the guide frame into the real or simulated subject.

Example 25. The method according to example 24, wherein advancing the guide frame into the real or simulated subject comprises transluminally advancing the guide frame into the real or simulated subject while the guide frame is constrained within a compressed state.

Example 26. The method according to any one of examples 20-25, wherein the method further comprises, subsequent to advancing the tool into the real or simulated subject, intracorporeally transitioning the teeth into a protruding state, such that the teeth protrude radially outward from a surface of the tool.

Example 27. The method according to example 26, wherein advancing the tool into the real or simulated subject comprises transluminally advancing the tool into the real or simulated subject while the teeth are in a retracted state in which the teeth lie flush with the surface of the tool.

Example 28. The method according to any one of examples 20-27, wherein: (i) the guide frame comprises struts that define windows therebetween, and/or (ii) driving the tool along the guide frame by rotating the tool while the radial teeth are engaged with the guide frame comprises driving the tool along the guide frame by rotating the tool while the radial teeth protrude into the windows of the guide frame.

Example 29. The method according to example 28, wherein the struts are arranged in a lattice-like framework, and/or wherein driving the tool along the guide frame comprises driving the tool along the lattice-like framework of the guide frame.

Example 30. The method according to any one of examples 20-29, wherein the method further comprises implanting an implant at the tissue, guided by the driving of the tool along the guide frame.

Example 31. The method according to example 30, wherein the implant is an elongate implant, and/or wherein implanting the implant at the tissue comprises implanting the elongate implant at the tissue.

Example 32. The method according to example 31, wherein: (i) the tissue is tissue of an annulus of a heart of the real or simulated subject, the annulus circumscribing an orifice of the valve, (ii) the elongate implant is an annuloplasty implant comprising a series of anchors, (iii) positioning the guide frame adjacent the tissue comprises positioning the guide frame through the orifice, and/or (iv) implanting the elongate implant at the tissue comprises implanting the annuloplasty implant at the tissue, by driving the tool around the guide frame, and/or intermittently pausing the driving to anchor an anchor of the series of anchors to the tissue.

Example 33. The method according to example 32, wherein implanting the annuloplasty implant at the tissue comprises, for the anchor of the series of anchors, engaging the anchor with an anchor driver, and/or delivering, via the tool, the anchor driver to the heart, and/or subsequently, using the anchor tool to anchor the anchor to the tissue.

Example 34. The method according to example 32, wherein anchors of the series of anchors are connected via a tether, and/or wherein implanting the implant along the tissue of the annulus comprises implanting the series of anchors along the tissue such that the tether extends along the annulus.

Example 35. The method according to example 34, wherein the method further comprises tensioning the tether to reduce a circumference of the annulus.

Example 36. The method according to example 35, wherein tensioning the tether to reduce the circumference of the annulus comprises contracting the guide frame radially inward while the guide frame is coupled to each anchor of the series.

Example 37. A system for use with tissue of a heart, the system comprising: (A) an implant comprising a series of anchors; and/or (B) a delivery assembly comprising: (i) a guide assembly that comprises: (1) a guide frame, and/or (2) multiple guides, a guide of the multiple guides extending longitudinally alongside an exterior of the guide frame to a coupling point, and/or (ii) an anchor tool, wherein the delivery assembly is configured to: (A) transluminally advance the guide frame into the heart, (B) within the heart, position the guide frame adjacent the tissue, (C) implant the implant while the guide frame remains positioned adjacent the tissue by, for an anchor of the series of anchors: (i) transluminally sliding the anchor tool over and along the guide, and/or alongside the guide frame, to an anchor site at the tissue adjacent the guide frame, and/or (ii) using the anchor tool, anchoring the anchor to the tissue at the anchor site, and/or (D) withdraw the guide frame from the heart.

Example 38. The system according to example 37, wherein: (A) the delivery assembly comprises a sheath that comprises: (i) a major lumen region, and/or (ii) a minor lumen region that has a smaller cross-sectional area than the major lumen region; (B) the delivery assembly is configured to transluminally advance the guide frame into the heart while: (i) the guide frame is disposed within the major lumen region of the sheath, and/or (ii) a control shaft coupled to the guide frame extends, from the guide frame, proximally through the major lumen region; and/or, (C) while the guide frame remains distally outside of the sheath and the control shaft remains coupled to the guide frame, the control shaft is shiftable laterally out of the major lumen region and into the minor lumen region, such that the anchor tool is advanceable through the major lumen region to the anchor site.

Example 39. The system according to any one of examples 37-38, wherein the anchor of the series of anchors is a helical anchor, and/or wherein the anchor tool is adapted to anchor the anchor to the tissue by screwing the helical anchor into the tissue.

Example 40. The system according to any one of examples 37-39, wherein the anchor of the series is a pledget anchor.

Example 41. The system according to any one of examples 37-40, wherein the guide is a wire.

Example 42. The system according to any one of examples 37-41, wherein the guide is a suture.

Example 43. The system according to any one of examples 37-42, wherein the guide frame comprises a framework of struts, the guide looping around a respective strut of the guide frame at the coupling point.

Example 44. The system according to any one of examples 37-43, wherein the guide: (i) extends, from a first end that is outside of the heart, to the heart, where the guide loops around the guide frame at the coupling point, and/or back out of the heart, and/or (ii) is intracardially decouplable from the guide frame by pulling a second end of the guide proximally.

Example 45. The system according to any one of examples 37-44, wherein: (i) the anchor tool comprises a lumen, and/or (ii) the delivery assembly is configured to slide the anchor tool over and along the guide by sliding the lumen over and along the guide.

Example 46. The system according to example 45, wherein: (i) the lumen is an oblique lumen at a distal end portion of the anchor tool, the tool having an intermediate portion proximal from the distal end portion, and/or (ii) the delivery assembly is configured to slide the lumen over and along the guide by sliding the oblique lumen distally along the guide while the intermediate portion is disposed alongside the guide.

Example 47. The system according to any one of examples 37-46, wherein the guide extends, from an extracorporeal end of the guide, transluminally through the delivery assembly and longitudinally alongside an exterior of the guide frame to the coupling point.

Example 48. The system according to example 47, wherein: (A) the delivery assembly comprises a sheath that comprises: (i) a primary lumen, (ii) multiple secondary lumens disposed alongside the primary lumen, and/or (iii) multiple slits, a slit of the multiple slits connecting a secondary lumen of the multiple secondary lumens to the primary lumen, (B) the guide is housed within the secondary lumen, and/or (C) the delivery assembly is configured to, for the anchor of the series of anchors, slide the anchor tool distally through the primary lumen such that the anchor tool pulls progressively distal portions of the guide laterally through the slit and into the primary lumen.

Example 49. The system according to example 48, wherein the multiple secondary lumens are arranged circumferentially around the primary lumen.

Example 50. The system according to any one of examples 37-49, wherein: (A) the anchor tool comprises: (i) an anchor catheter, and/or (ii) an anchor driver advanceable through a lumen of the anchor catheter, (B) the delivery assembly is configured to slide the anchor tool over and along the guide by sliding the anchor catheter over and along the guide, and/or (C) the delivery assembly is configured to anchor the anchor to the tissue at the anchor site using the anchor driver while the anchor driver extends through the lumen of the anchor catheter.

Example 51. The system according to example 50, wherein: (i) the lumen of the anchor catheter is a driver lumen and is eccentric, (ii) the anchor catheter further comprises a guide lumen that is eccentric, and/or (iii) the delivery assembly is configured to slide the anchor catheter over and along the guide by sliding the guide lumen over and along the guide.

Example 52. The system according to any one of examples 37-51, wherein the delivery assembly is configured to position the guide frame adjacent the tissue by expanding the guide frame within the heart.

Example 53. The system according to example 52, wherein the delivery assembly is configured to position the guide frame adjacent the tissue by expanding the guide frame within the heart until the guide frame abuts the tissue.

Example 54. The system according to any one of examples 37-53, wherein the delivery assembly is configured to: (i) position the guide frame adjacent the tissue such that the coupling point of the guide is distal to the tissue, and/or (ii) the delivery assembly is configured to slide the anchor tool distally over and along the guide until the anchor tool abuts the tissue at the anchor site, proximal from the coupling point.

Example 55. The system according to example 54, wherein the delivery assembly is configured to sandwich the guide between the guide frame and the tissue, such that the tissue obstructs the anchor tool from reaching the coupling point.

Example 56. The system according to example 54, wherein: (i) the tissue is tissue of an annulus of a valve of the heart, the annulus circumscribing an orifice of the valve, (ii) the implant is an annuloplasty implant comprising the series of anchors, (iii) the delivery assembly is configured to position the guide frame through the orifice such that the multiple guides extend through the orifice alongside the exterior of the guide frame, and/or (iv) for the anchor of the series of anchors, the delivery assembly is configured to slide the tool alongside the guide frame until the tool abuts the annulus.

Example 57. The system according to any one of examples 37-56, wherein the implant is an elongate implant, and/or wherein the delivery assembly is configured to guide the implantation of the elongate implant circumferentially around the guide frame by anchoring anchors of the series of anchors to corresponding anchor sites.

Example 58. The system according to example 57, wherein: (i) the tissue is tissue of an annulus of an atrioventricular valve of the heart, (ii) the valve has an atrium upstream of the valve, and/or a ventricle downstream of the valve, (iii) the elongate implant is an annuloplasty implant comprising the series of anchors, and/or (iv) the delivery assembly is configured to guide the implantation of the elongate implant circumferentially around the guide frame such that the implant extends along the annulus.

Example 59. The system according to example 58, wherein: (i) the anchor of the series of anchors is a toggle anchor, and/or (ii) the delivery assembly is configured to anchor the anchor by advancing the anchor, from the atrium through the tissue of the annulus and into the ventricle.

Example 60. The system according to example 58, wherein the implant further comprises a tether that connects anchors of the series of anchors, and/or wherein the tool is configured to implant the implant such that the tether extends along the annulus.

Example 61. The system according to example 60, wherein the delivery assembly is configured to reduce a circumference of the annulus by contracting the guide frame while the anchors remain anchored to the annulus.

Example 62. The system according to example 60, wherein the delivery assembly is configured to reduce a dimension of the annulus by applying tension to the tether while the anchors remain anchored to the annulus.

Example 63. The system according to example 62, wherein the delivery assembly is configured to maintain the annulus at the reduced dimension by locking the tension in the tether.

Example 64. The system according to any one of examples 37-63, wherein: (i) the guide assembly further comprises multiple flaps spaced circumferentially along the exterior of the guide frame, (ii) the guide is coupled to the guide frame via a flap of the multiple flaps, and/or (iii) the flap is intracorporeally deflectable away from the guide frame in a manner that spaces the guide from the guide frame.

Example 65. The system according to example 64, wherein, the anchor, the delivery assembly is configured to anchor the anchor to the tissue at the anchor site by delivering the anchor through the flap and into the tissue.

Example 66. The system according to example 64, wherein the flap is deflectable from a retracted position, in which the flap lies against the exterior of the guide frame, towards a deployed position, in which the flap lies against the tissue.

Example 67. The system according to example 66, wherein the flap is transitionable to the deployed position by pushing the guide distally.

Example 68. The system according to example 64, wherein the flap is attached to the guide frame via a connecting wire that: (i) weaves in and out of the guide frame, and/or (ii) is exposed out of the guide frame and loops through the flap.

Example 69. The system according to example 68, wherein the flap is intracardially disengageable from the guide frame by retracting the connecting wire, such that the connecting wire unloops from the flap.

Example 70. The system according to example 69, wherein the delivery assembly is configured to: (i) for the anchor of the series of anchors, anchor the anchor to the tissue in a manner that couples the anchor to the flap, (ii) retract the connecting wire to disengage the guide frame from the flap, and/or (iii) withdraw the guide frame from the heart such that the flap remains coupled to the anchor within the heart.

Example 71. The system according to example 70, wherein: (i) the anchor comprises a toggle anchor, the toggle anchor comprising a toggle, and/or a cord that extends away from the toggle, (ii) the delivery assembly is configured to anchor the anchor by advancing the toggle, from a first chamber of the heart, through the flap and the tissue, into a second chamber of the heart, such that the cord extends from the toggle in the second chamber, through the tissue and the flap, to an anchor head that is disposed against the flap within the first chamber, (iii) the delivery assembly is configured to retract the connecting wire to disengage the guide frame from the flap, and/or (iv) the delivery assembly is configured to withdraw the guide frame from the heart such that the flap comprises a first component of the toggle anchor, and/or the anchor defines a second component of the toggle anchor.

Example 72. A method for use with tissue of a heart, the method comprising: (i) transluminally advancing a guide frame into the heart while multiple guides extend longitudinally alongside an exterior of the guide frame to respective coupling points on the guide frame, (ii) within the heart, positioning the guide frame adjacent the tissue, (iii) while the guide frame remains adjacent the tissue, sliding an anchor tool over and along a guide of the multiple guides, and/or alongside the guide frame, to an anchor site at the tissue that is adjacent the guide frame, (iv) subsequently, using the anchor tool, anchoring an anchor to the tissue at the anchor site, and/or (v) subsequently, withdrawing the guide frame from the heart.

Example 73. The method according to example 72, wherein the anchor is a helical anchor, and/or wherein anchoring the anchor to the tissue comprises screwing the helical anchor into the tissue.

Example 74. The method according to any one of examples 72-73, wherein the anchor is a pledget anchor, and/or wherein anchoring the anchor to the tissue comprises anchoring the pledget anchor to the tissue.

Example 75. The method according to any one of examples 72-74, wherein the guide is at least one of a wire and a suture.

Example 76. The method according to any one of examples 72-75, wherein the guide frame comprises a framework of struts, the guide looping around a strut of the guide frame at a coupling point, and/or wherein positioning the guide frame adjacent the tissue comprises positioning the guide frame adjacent the tissue, such that the coupling point is positioned distally to the tissue.

Example 77. The method according to any one of examples 72-76, wherein: (i) the guide extends, from a first end that is outside of the heart, to the heart, where the guide loops around the guide frame at a coupling point, and/or back out of the heart, and/or (i) the method further comprises intracardially decoupling the guide from the guide frame by pulling an end of the guide proximally.

Example 78. The method according to any one of examples 72-77, wherein: (i) transluminally advancing the guide frame into the heart comprises transluminally advancing the guide frame into the heart via a major lumen region of a sheath, with a control shaft coupled to the guide frame, wherein the control shaft extends from the guide frame, proximally through the major lumen region, (ii) positioning the guide frame adjacent the tissue comprises delivering the guide frame distally outside of the sheath and positioning the guide frame adjacent the tissue, (iii) while the guide frame remains distally out of the sheath, and/or the control shaft remains coupled to the guide frame, the method further comprises shifting the control shaft laterally out of the major lumen region and into a minor lumen region of the sheath, the minor lumen region having a smaller cross-sectional area than the major lumen region, and/or (iv) sliding the anchor tool over and along the guide comprises sliding the anchor tool through the major lumen region, over and along the guide.

Example 79. The method according to example 78, wherein shifting the control shaft laterally out of the major lumen region and into the minor lumen region comprises sliding the anchor tool through the major lumen region, such that the control shaft is pushed out of the major lumen region and into the minor lumen region.

Example 80. The method according to any one of examples 72-79, wherein: (i) the anchor tool comprises a lumen, and/or (ii) sliding the anchor tool over and along the respective one of the guides comprises sliding the lumen over and along the respective one of the guides.

Example 81. The method according to example 80, wherein: (i) the lumen is an oblique lumen at a distal end portion of the anchor tool, the anchor tool having an intermediate portion proximal from the distal end portion, and/or (ii) sliding the lumen over and along the respective the guide comprises sliding the oblique lumen distally over and along the guide while the intermediate portion is disposed alongside the guide.

Example 82. The method according to any one of examples 72-81, wherein: (A) sliding the anchor tool over and along the guide comprises sliding the anchor tool over and along the guide while the anchor tool is housed within a sheath, wherein the sheath comprises: (i) a primary lumen, (ii) multiple secondary lumens disposed alongside the primary lumen, the guide being housed within a secondary lumen, and/or (iii) multiple slits, a slit of the multiple slits connecting the secondary lumen to the primary lumen, and/or (B) sliding the anchor tool over and along the guide comprises sliding the anchor tool distally within the primary lumen, such that the anchor tool pulls progressively distal portions of the guide laterally through the slit and into the primary lumen.

Example 83. The method according to example 82, wherein the multiple secondary lumens are arranged circumferentially around the primary lumen.

Example 84. The method according to any one of examples 72-83, wherein: (A) the anchor tool includes: (i) an anchor catheter, and/or (ii) an anchor driver extendable through a lumen of the anchor catheter, (B) sliding the anchor tool over and along the guide comprises sliding the anchor catheter over and along the guide, and/or (C) anchoring the anchor to the tissue comprises using the anchor driver, while the anchor driver is extended within a lumen of the anchor catheter.

Example 85. The method according to example 84, wherein: (i) the lumen of the anchor catheter is a driver lumen and is eccentric, (ii) the anchor catheter further comprises a guide lumen that is eccentric, and/or (iii) sliding the anchor tool over and along the guide comprises sliding the guide lumen over and along the guide.

Example 86. The method according to any one of examples 72-85, wherein positioning the guide frame adjacent the tissue comprises expanding the guide frame within the heart until the guide frame abuts the tissue.

Example 87. The method according to example 86, wherein expanding the guide frame within the heart until the guide frame abuts the tissue comprises expanding the guide frame within the heart until the guide frame presses against the tissue.

Example 88. The method according to any one of examples 72-87, wherein: (i) positioning the guide frame adjacent the tissue comprises positioning the guide frame adjacent the tissue such that the respective coupling points are positioned distally to the tissue, and/or (ii) sliding the anchor tool over and along the guide comprises sliding the anchor tool over and along the guide until the anchor tool abuts the tissue at the anchor site, the anchor site being proximal from a coupling point associated with the guide.

Example 89. The method according to example 88, wherein positioning the guide frame adjacent the tissue comprises positioning the guide frame adjacent the tissue in a manner that sandwiches at the guide between the guide frame and the tissue, such that the tissue obstructs the anchor tool from reaching the coupling point.

Example 90. The method according to example 88, wherein: (i) the tissue is tissue of an annulus of a valve of the heart, the annulus circumscribing an orifice of the valve, (ii) the anchor is an anchor of an annuloplasty implant, (iii) positioning the guide frame adjacent the tissue comprises positioning the guide frame through the orifice such that the guide extends through the orifice alongside the exterior of the guide frame, and/or (iv) sliding the anchor tool over and along the guide comprises sliding the anchor tool over and along the guide until the anchor tool abuts the annulus.

Example 91. The method according to any one of examples 72-90, wherein: (A) the anchor is an anchor of a series of anchors of an elongate implant, (B) the method further comprises guiding the implantation of the elongate implant circumferentially around the guide frame by, for each anchor of the series of anchors: (i) sliding the anchor tool over and along a respective one of the guides, and/or alongside the guide frame, to a respective site at the tissue that is adjacent the guide frame, and/or (ii) anchoring each anchor to respective anchor sites along the tissue.

Example 92. The method according to example 91, wherein: (i) the tissue is tissue of an annulus of an atrioventricular valve of the heart, (ii) the valve has an atrium upstream of the valve, and/or a ventricle downstream of the valve, (iii) the elongate implant is an annuloplasty implant comprising the series of anchors, and/or (iv) guiding the implantation of the elongate implant circumferentially around the guide frame comprises guiding the implantation of the annuloplasty implant circumferentially around the guide frame such that the annuloplasty implant extends along the annulus.

Example 93. The method according to example 92, wherein: (i) the anchor is a toggle anchor, and/or (ii) anchoring the anchor of the series of anchors to the respective anchor sites along the tissue comprises, for each anchor of the series, advancing the anchor from the atrium through the tissue of the annulus and into the ventricle.

Example 94. The method according to example 92, wherein the annuloplasty implant further comprises a tether that connects anchors of the series of anchors, and/or wherein guiding the implantation of the annuloplasty implant circumferentially around the guide frame comprises guiding the implantation of the annuloplasty implant circumferentially around the guide frame such that the tether extends along the annulus.

Example 95. The method according to example 94, wherein the method further comprises reducing a dimension of the annulus by applying tension to the tether while the anchors remain anchored to the annulus.

Example 96. The method according to example 95, wherein the method comprises maintaining the annulus at the reduced dimension by locking the tension in the tether.

Example 97. The method according to example 95, wherein applying tension to the tether comprises applying tension to the tether by contracting the guide frame while the anchors remain anchored to the annulus.

Example 98. The method according to any one of examples 72-97, wherein: (i) advancing the guide frame into the heart comprises advancing the guide frame into the heart while multiple flaps are spaced circumferentially along the exterior of the guide frame, (ii) the guide is coupled to the guide frame via a flap of the multiple flaps, and/or (iii) the method comprises, subsequently to positioning the guide frame adjacent the tissue, and/or prior to sliding the anchor tool over and along the guide, deflecting the flap away from the guide frame in a manner that spaces the guide away from the guide frame.

Example 99. The method according to example 98, wherein, anchoring the anchor to the tissue comprises anchoring the anchor adjacent the flap and into the tissue.

Example 100. The method according to example 99, further comprising, subsequent to anchoring the anchor adjacent the flap, withdrawing the flap from the heart.

Example 101. The method according to example 98, wherein deflecting the flap away from the guide frame comprises deflecting the flap, from a retracted position in which the flap lies against the exterior of the guide frame, towards a deployed position in which the flaps lie against the tissue.

Example 102. The method according to example 100, wherein deflecting the flap away from the guide frame comprises pushing its corresponding guide distally.

Example 103. The method according to example 98, wherein: (A) the multiple flaps are attached to the guide frame via a connecting wire that: (i) weaves in and out of the guide frame, and/or (ii) at a flap of the multiple flaps, the connecting wire is exposed out of the guide frame and loops through the flap, and/or (B) sliding the anchor tool over and along the guide comprises sliding the anchor tool over and along the guide while the flap is connected to the guide frame via the connecting wire.

Example 104. The method according to example 103, wherein the method further comprises intracardially disengaging the multiple flaps from the guide frame by retracting the connecting wire, such that the connecting wire unloops from the multiple flaps.

Example 105. The method according to example 104, wherein, anchoring the anchor to the tissue comprises delivering the anchor through the flap and into the tissue.

Example 106. The method according to example 105, wherein: (i) anchoring the anchor to the tissue comprises anchoring the anchor to the tissue in a manner that couples the anchor to the flap, and/or (ii) the method further comprises, subsequent to disengaging the multiple flaps from the guide frame, withdrawing the guide frame from the heart such that the flap remains coupled to the anchor within the heart.

Example 107. The method according to example 106, wherein: (i) the anchor comprises a toggle anchor, the toggle anchor comprising a toggle, and/or a cord that extends away from the toggle, (ii) anchoring the anchor to the tissue comprises advancing the toggle, from a first chamber of the heart, through the flap and the tissue, into a second chamber of the heart, such that the cord extends from the toggle in the second chamber, through the tissue, to the flap in the first chamber, and/or (iii) withdrawing the guide frame from the heart comprises withdrawing the guide frame from the heart such that the flap comprises a first component of the toggle anchor, and/or the anchor comprises a second component of the toggle anchor.

Example 108. A method for use with a heart of a real or simulated subject, the method comprising: (A) transluminally advancing a distal end of a sheath toward the heart, the sheath comprising a lumen therethrough, the lumen having, at least at the distal end: (i) a major lumen region, and/or (ii) a minor lumen region that has a smaller cross-sectional area than the major lumen region; (B) positioning the distal end of the sheath within the heart while a control shaft extends distally through the major lumen region; (C) using the control shaft, pushing a device, coupled to a distal end of the control shaft, distally out of the major lumen region to become disposed distally outside of the sheath, and/or (D) while the device remains distally outside of the sheath and coupled to the control shaft, shifting the control shaft laterally out of the major lumen region and into the minor lumen region.

Example 109. The method according to example 108, wherein the method further comprises, subsequent to pushing the device distally out of the sheath, expanding the device within the heart.

Example 110. The method according to any one of examples 108-109, wherein: (i) the device is a first device, and/or (ii) the method further comprises, while the first device remains distally outside of the sheath, advancing a second device through the major lumen region and distally out of the sheath.

Example 111. The method according to example 110, wherein shifting the control shaft laterally out of the major lumen region and into the minor lumen region comprises advancing the second device through the major lumen region such that the second device progressively pushes the control shaft out of the major lumen region and into the minor lumen region.

Example 112. The method according to example 110, wherein advancing the second device through the major lumen region and distally out of the sheath comprises advancing the second device through the major lumen region and distally out of the sheath while the second device is coupled to the first device.

Example 113. The method according to example 110, wherein: (i) the first device is a guide frame, and/or (ii) advancing the second device distally out of the sheath comprises advancing the second device distally out of the sheath and towards a site at the heart, guided by the guide frame.

Example 114. The method according to example 113, wherein the second device is an anchor tool, and/or the method further comprises, while the guide frame remains distally outside of the sheath and the control shaft remains in the minor lumen region, anchoring an anchor to the tissue, by: (i) advancing the anchor tool through the major lumen region, while the anchor tool is engaged with the anchor, towards tissue of the heart, and/or (ii) using the anchor tool to anchor the anchor to the tissue.

Example 115. Apparatus for use with a first device and a second device, the apparatus comprising: (A) a sheath for advancing into vasculature of a real or simulated subject, the sheath defining a lumen therethrough, the lumen, at least at a distal end of the sheath, comprising: (i) a major lumen region, and/or (ii) a minor lumen region that has a smaller cross-sectional area than the major lumen region; wherein: (A) the first device is: (i) transluminally advanceable through the sheath while the first device is disposed within the major lumen region of the sheath and a control shaft extends, from the first device, proximally away from the first device through the major lumen region, and/or (ii) deliverable out of the distal end of the sheath, such that the control shaft extends from the first device, proximally through the major lumen region, and/or (B) the second device is transluminally advanceable through the major lumen region while the first device is disposed out of the distal end of the sheath and the control shaft remains coupled to the first device, by shifting the control shaft laterally out of the major lumen region and into the minor lumen region to vacate the major lumen region for the second device.

Example 116. A method for use with tissue of a heart, the method comprising: (i) transluminally advancing a guide frame into the heart while a guide extends longitudinally alongside an exterior of the guide frame to a coupling point on the guide frame; (ii) within the heart, positioning the guide frame adjacent the tissue such that the coupling point is distally beyond the tissue, and/or the guide is sandwiched between the guide frame and the tissue; and/or (iii) while the guide remains sandwiched between the guide frame and the tissue, positioning an implant at the tissue by advancing the implant along the guide alongside the guide frame until the implant abuts the tissue.

Example 117. A system for use with tissue of a heart, the system comprising: (A) an implant comprising a series of anchors; and/or (B) a guide assembly that comprises: (i) a guide frame, and/or (ii) multiple guides, a guide of the multiple guides extending alongside an exterior of the guide frame to a coupling point, and/or an anchor tool, wherein the guide assembly is configured to be transluminally advance into the heart and be positioned adjacent the tissue, such that the implant can be implanted in the tissue while the guide frame is positioned adjacent the tissue by: (i) transluminally sliding the anchor tool over and along the guide, and/or alongside the guide frame, to an anchor site at the tissue adjacent the guide frame, and/or (i) using the anchor tool, secure one or more anchors of the series at the anchor site.

Example 118. A system according to any of the above examples, in which the system, the delivery assembly, the guide assembly, the guide frame, the tool, the implant, and/or the anchor is sterilized.

1. A method for use with tissue of a heart, the method comprising:
transluminally advancing a frame into the heart while a wire extends alongside an exterior of the frame;
within the heart, positioning the frame adjacent the tissue such that the wire becomes positioned against the tissue; and
while the wire remains positioned against the tissue, transluminally advancing a tube over and along the wire and alongside the frame to the tissue;
while the tube remains positioned alongside the frame, advancing an anchor along the tube to the tissue; and
anchoring the anchor into the tissue.

2. The method according to claim 1, wherein positioning the frame adjacent the tissue comprises expanding the frame within the heart until the frame presses against the tissue.

3. The method according to claim 1, wherein:
the tissue is tissue of an annulus of a valve of the heart, the annulus circumscribing an orifice of the valve,
positioning the frame adjacent the tissue comprises positioning the frame within the orifice such that the frame presses against the annulus, and
anchoring the anchor into the tissue comprises anchoring the anchor into annular tissue.

4. The method according to claim 1, wherein the anchor is a helical anchor, and wherein anchoring the anchor to the tissue comprises screwing the helical anchor into the tissue.

5. The method according to claim 1, wherein anchoring the anchor into the tissue comprises anchoring the anchor into the tissue adjacent the frame.

6. The method according to claim 1, wherein the anchor is a component of an implant, and wherein the method comprises using the tube to guide the implantation of the implant circumferentially around the frame.

7. The method according to claim 6, wherein the implant includes a tether, and wherein the method further comprises reducing a dimension of the tissue by applying tension to the tether while (i) the tether extends circumferentially along the tissue, and (ii) the anchor remains anchored to the tissue.

8. The method according to claim 7, wherein the method further comprises maintaining the tissue at the reduced dimension by locking the tension in the tether.

9. The method according to claim 7, wherein applying tension to the tether comprises applying tension to the tether by contracting the frame while the anchor remain anchored to the tissue.

10. The method according to claim 1, wherein a fabric is attached to the frame, and wherein anchoring the anchor into the tissue comprises anchoring the anchor through the fabric and into the tissue.

11. The method according to claim 10, wherein transluminally advancing the frame into the heart comprise transluminally advancing the frame into the heart while the fabric is flush against the frame, and wherein the fabric is intracorporeally deflectable away from the frame.

12. A method for use with tissue of a heart, the method comprising:
transluminally advancing a frame into the heart while a wire extends alongside an exterior of the frame;
within the heart, positioning the frame adjacent the tissue such that the wire becomes positioned against the tissue; and
while the wire remains positioned against the tissue, transluminally advancing a tube over and along the wire and alongside the frame to the tissue;
subsequently securing an implant to the tissue circumferentially around the frame by:
transluminally advancing an anchor along the tube, alongside the frame to the tissue; and
anchoring the anchor to the tissue.

13. A system for use with tissue of a heart, the system comprising:
a frame;
a wire extending alongside an exterior of the frame;
a tube;
an anchor; and
a delivery assembly, configured to:
transluminally advance the frame into the heart,
within the heart, position the frame adjacent the tissue,
while the frame remains positioned adjacent the tissue, transluminally slide the tube over and along the wire and alongside the frame to an anchor site at the tissue adjacent the frame, and
anchor the anchor to the tissue at the anchor site.

14. The system according to claim 13, wherein the frame is adapted to self-expand within the heart such that the frame presses against the tissue.

15. The system according to claim 13, wherein the anchor is a helical anchor.

16. The system according to claim 13, wherein the anchor is a component of an implant, and wherein the delivery assembly is adapted to guide the implantation of the implant circumferentially around the frame.

17. The system according to claim 16, wherein the implant includes a tether, and wherein the delivery assembly is adapted to apply tension to the tether while (i) the tether extends circumferentially along the tissue, and (ii) the anchor remains anchored to the tissue.

18. The system according to claim 17, wherein the implant further comprises a lock, lockable onto the tether in a manner that maintains the tension int the tether.

19. The system according to claim 13, wherein a fabric is attached to the frame, and wherein the delivery assembly is adapted to anchor the anchor through the fabric and into the tissue.

20. The system according to claim 19, wherein the delivery assembly is adapted to transluminally advance the frame into the heart while the fabric is flush against the frame, and wherein the fabric is intracorporeally deflectable away from the frame.

21. A system for use with tissue of a cardiovascular system of a subject, the system comprising:
a delivery assembly comprising:
a guide frame, the delivery assembly configured to position the guide frame adjacent the tissue, and
a tool comprising radial teeth adapted to engage the guide frame such that rotation of the tool drives the tool along the guide frame, the tool being configured to guide a treatment of the tissue via the driving of the tool along the guide frame.

22. Apparatus for use with a first device and a second device, the apparatus comprising: a sheath for advancing into vasculature of a subject, the sheath defining a lumen therethrough, the lumen, at least at a distal end of the sheath, comprising:
a major lumen region, and
a minor lumen region that has a smaller cross-sectional area than the major lumen region;
wherein:
the first device is:
transluminally advanceable through the sheath while the first device is disposed within the major lumen region of the sheath and a control shaft extends, from the first device, proximally away from the first device through the major lumen region, and
deliverable out of the distal end of the sheath, such that the control shaft extends from the first device, proximally through the major lumen region, and
the second device is transluminally advanceable through the major lumen region while the first device is disposed out of the distal end of the sheath and the control shaft remains coupled to the first device, by shifting the control shaft laterally out of the major lumen region and into the minor lumen region to vacate the major lumen region for the second device.
